(19) United States
(12) Patent Application Publication
(10) Pub. No.: US 2011/0130827 A1
(43) Pub. Date: Jun. 2, 2011

(54) VASCULAR PROTECTIVE DEVICE

(75) Inventors: WILLIAM G. BRODBECK, Hudson, OH (US); YOONSU CHOI, Atlanta, GA (US); VERNON H. MERRITT, Duluth, GA (US); JASMINE D. PATEL, Shaker Hts.,, OH (US); UDAYAN PATEL, San Jose, CA (US); MICHAEL J. WIGGINS, Cleveland, OH (US)

(73) Assignee: ICON Medical Corp.

(21) Appl. No.: 13/021,929

(22) Filed: Feb. 7, 2011

Related U.S. Application Data
(63) Continuation of application No. 11/699,586, filed on Jan. 25, 2007.
(60) Provisional application No. 60/763,556, filed on Jan. 31, 2006.

(51) Int. Cl. A61F 2/82

(52) U.S. Cl. 623/1.15

(57) ABSTRACT

A medical device to protect and/or heal a diseased and/or injured area in the body passageway.

The present invention is a continuation of U.S. application Ser. No. 11/699,586 filed Jan. 15, 2007, which in turn claims priority on U.S. Provisional Patent Application Ser. No. 60/763,556 filed Jan. 31, 2006, which is incorporated herein by reference.

The present invention relates generally to medical devices, and more particularly to a medical device that can be inserted into a body passageway to facilitate in repairing and/or treating a diseased portion of the body passageway, and still more particularly to a medical device that can be inserted into a blood vessel to facilitate the repair and/or treatment of vulnerable plaque, dissections, and/or vascular malformations (e.g., Aneurysms and Arterial Venular Malformations) in the blood vessel.

### BACKGROUND OF THE INVENTION

Vulnerable plaque is a type of fatty buildup in a blood vessel (e.g., arteries, etc.) thought to be caused by inflammation. The plaque is covered by a thin, fibrous cap that upon rupture may lead to the formation of a blood clot and, ultimately, occlusion of the blood vessel. Plaque rupture most often occurs in smaller blood vessels such as, but not limited to, the coronary arteries, which supply blood to the heart muscle. The occlusion of a coronary artery can lead to a heart attack.

Medical devices such as, but not limited to stents, angioplasty balloons, etc., are commonly used to improve the flow of blood through a blood vessel. Blood vessels that include plaque have obstructed blood flow through the area of plaque. This obstructed blood flood can cause damage to the blood vessel, damage to one or more organs being supplied blood by the blood vessel, unacceptably increased blood pressure in one or more regions of the body, etc. The medical devices used to restore proper blood flow through the blood vessel are typically inserted into the diseased portion of the blood vessel and then expanded so as to partially compress or flatten the plaque against the wall of the blood vessel, thereby improving the flow of blood through the blood vessel. Due to the relatively fragile nature of some plaque and/or the size of the plaque, the compression or flattening of the plaque by a medical device can cause the plaque to rupture, which in turn can result in undesirable consequences.

Vessel dissections are often created during interventional procedures in the vasculature and/or occur as a result of disease. Dissections are characterized as a separation in the intimal layer of the vessel wall protruding into or blocking the flow of blood within the vessel.

Medical devices such as, but not limited to stents, angioplasty balloons, etc., are commonly used to treat dissections. Blood vessels that include dissections are at risk of rupture causing damage to the blood vessel, damage to one or more organs being supplied blood by the blood vessel, unacceptably increased blood pressure in one or more regions of the body, etc. The medical devices used to treat dissections in blood vessel are typically inserted into the diseased portion of the blood vessel and then expanded so as to partially compress, flatten, or hold the dissected portion of the vessel thereby improving the flow of blood through the blood vessel and reducing the risk. Due to the relatively fragile nature of dissected vessel, the compression or flattening by a medical device can cause the vessel to rupture, which in turn can result in undesirable consequences.

Aneurysms are a weakening in the vessel wall resulting in a protrusion from the vessel. Similarly Arterial Venular Malformations (AVMs) are characterized as a lack of vascular structure between the arterial and venular circulation resulting in a network of high pressure in vessels without the ability to regulate the pressure difference between the arterial and venular circulation. Both Aneurysms and AVM can occur due to disease, injury, or congenital defect and can result in bleeding within the cerebral tissue.

Medical devices such as, but not limited to stents, angioplasty balloons, detachable coils, and embolic agents, etc., are commonly used to treat Aneurysms or AVMs by excluding the malformation from the circulation. Re-occurrence of the disease commonly occurs as a result of blood flow into the treated malformation. Devices able to completely exclude the malformation and ensure blood flow is directed away from the malformations have significant advantage.

In view of the current state of technology with regard to medical devices for use in a blood vessel, there is a need for a medical device that safely treats blood vessels that include vulnerable plaque, dissections, and/or vascular malformations and which medical device reduces the occurrence of undesirable consequences during the use of the medical device.

### SUMMARY OF THE INVENTION

The present invention is directed to a medical device designed to be inserted into a body passageway to facilitate in repairing repair and/or treating a diseased portion of the body passageway such as, but not limited to, vulnerable plaque in a blood vessel. As used herein, the term body passageway is defined to be any passageway or cavity in a living organism (e.g., bile duct, bronchial tube, nasal cavity, blood vessel, heart, esophagus, trachea, stomach, fallopian tube, uterus, ureter, urethra, the intestines, lymphatic vessel, nasal passageway, eustachian tube, acoustic meatus, etc.). For vascular applications, the term body passageway primarily refers to blood vessels and chambers in the heart. The medical device of the present invention is designed to at least partially anchor itself at or near one or more ends of the medical device and to provide a protective and/or supportive skin (e.g., thin film, thin sheet, etc.) over at least a portion of a diseased area of the body passageway and at the same time minimize or avoid injury, damage and/or rupture of the diseased area of the body passageway. In one non-limiting embodiment of the invention, the medical device is designed to provide a protective and/or supportive skin (e.g., thin film, thin sheet, etc.) at least partially over a diseased area in a body passageway so as to inhibit or prevent susceptible areas of the diseased area from naturally rupturing and/or rupturing during the insertion of the medical device in the body passageway. In one non-limiting aspect of this embodiment, the medical device is designed to provide a protective and/or supportive skin (e.g., thin film, thin sheet, etc.) at least partially over a diseased area (e.g., area containing plaque, etc.) in a blood vessel so as to inhibit or prevent the diseased area from naturally rupturing and/or rupturing during the insertion of the medical device in the blood vessel, which rupturing could lead to a blockage of the blood vessel. The diseased area of a blood vessel is typically in a weakened state, thus the diseased area can be highly susceptible to injury, damage and/or rupture when a medical device is inserted into the diseased area. The medical device of the present invention is designed to provide protection to these diseased areas and to provide protection to such areas without unduly aggravating the area so as to result in injury, damage and/or rupture to the diseased area. The design of the medical device of the present invention is thus a significant advancement over prior art devices. In another and/or alternative non-limiting aspect of this embodiment, the medical device is designed to provide one or more anchoring members at or near one or more ends of the medical device. These one or more anchoring members are designed to at least partially maintain the position of the medical device in a body passageway once the medical device has been properly inserted and positioned in the diseased area of the body passageway. In one non-limiting design, one or more anchoring members can be designed to facilitate in maintaining the protective and/or supportive skin (e.g., thin film, thin sheet, etc.) of the medical device on or in close proximity to the diseased area of the body passageway. In one non-limiting aspect of the present invention, at least one of the anchoring members are designed to be positioned distally or proximally to the diseased area in the body passageway so that little or no direct contact and/or force is applied by such anchoring members to the diseased area on the body passageway. As such, the one or more anchoring members can be used to at least partially anchor the medical device in position in the body passageway and to apply little or no stress on the diseased area of the body passageway. In another and/or alternative non-limiting aspect of the present invention, at least one anchoring member is designed to be positioned distally to the diseased area in the body passageway and at least one other anchoring member is designed to be positioned proximally to the diseased area in the body passageway so that both anchoring members are spaced from the diseased area in the body passageway. The spacing of two anchoring members from one another on the medical device is typically dependant on, but not limited to, the type of body passageway the medical device is to be inserted into, the size of the diseased area in the body passageway, and/or the location of the diseased area in the body passageway. When the medical device is to be used in a blood vessel, the spacing of the two end anchoring members from one another is typically less than about 150 mm, typically about 1-100 mm, more typically about 1-50 mm, and even more typically about 5-35 mm; however, it will be appreciated that other separation distances can be used.

In one non-limiting aspect of the present invention, the protective and/or supportive skin of the medical device is at least partially in the form of a thin sheet or film. As used herein thin sheet and thin film are used interchangeably. The thin sheet is designed and formed of a material to at least partially protect and/or support one or more portions of a diseased area (e.g., vulnerable plaque, etc.) in a body passageway. The thin sheet can be fully or partially formed of biostable or bioabsorbable materials. The thin sheet can be formed of one or more layers of material. The thin sheet can be formed of a uniform material throughout the thin sheet, or portions of the thin sheet can be different from other portions of the thin sheet (e.g., top surface of sheet having a different composition from bottom surface of the sheet, top sheet layer having a different composition from one or more other sheet layers, etc.). In one non-limiting embodiment of the invention, a majority of the thin sheet of material has an average thickness of less than about 2 mm when the medical device is used in a blood vessel. As can be appreciated, the thin sheet can have other thicknesses when the medical device is designed for use in body passageways other than blood vessels. In one non-limiting aspect of this embodiment, the average thickness of a majority of the thin sheet of material is less than or equal to about 1 mm. In another and/or alternative non-limiting aspect of this embodiment, the average thickness of the complete thin sheet of material is less than or equal to about 1 mm. In still another and/or alternative non-limiting embodiment of the invention, the thickness of the thin sheet of material can be uniform or vary in different regions of the sheet of material. In one non-limiting aspect of this embodiment, the thickness of the thin sheet of material is substantially uniform. In still another and/or alternative non-limiting aspect of this embodiment, the thin sheet of material can be absent essentially any holes or openings (i.e., solid sheet, etc.), or include one or more holes or openings (e.g., mesh designs, sheet with holes, etc.). In yet another and/or alternative non-limiting aspect of this embodiment, the thin sheet of material can be at least partially formed of a porous material or a non-porous material. In another and/or alternative non-limiting embodiment of the invention, the thin sheet is less rigid and/or more flexible than one or all of the anchoring members of the medical device. The thin sheet of material is used in part to form a protective layer over all or a portion of a diseased area in the body passageway, whereas the anchoring members are designed to engage an inner surface of the body passageway to at least partially anchor the medical device in the body passageway. The anchoring function of the anchoring members typically requires the anchoring members to have a strength and rigidity to enable the anchoring members to maintain the medical device in position in the body passageway. As such, the anchoring members are typically designed to maintain an expanded state when the anchoring members have been expanded and are at least partially anchoring the medical device in the body passageway. The thin sheet typically has little, if any, anchoring function, thus can have significantly different properties from the anchoring members; however, this is not required. The thin film or sheet can be secured to one or portions of the medical device. In one non-limiting arrangement, the thin film or sheet is at least partially secured to one or more of the anchoring members of the medical device. In another or additional arrangement, thin film or sheet is at least partially secured to one or more of the cross members of the medical device. The thin film or sheet can be at least partially secured to one or more of the anchoring members and/or cross members of the medical device on the top surface, bottom surface and/or side surfaces of the anchoring members and/or cross members.

In another and/or alternative non-limiting aspect of the present invention, the protective and/or supportive skin (e.g., thin film, thin sheet, etc.) of the medical device and/or one or more of the anchoring members can be at least partially formed of one or more polymers, metals (e.g., aluminum, barium, bismuth, calcium, carbon, cobalt, copper, chromium, depleted radioactive elements, gold, iron, lead, molybdenum, magnesium, nickel, niobium, platinum, rare earth metals, rhenium, silver, tantalum, titanium, tungsten, vanadium, yttrium, zinc, zirconium, and/or alloys thereof [e.g., stainless steel, nitinol, Cr—Co, Mo—Re, Ta—W, Mg—Zr, Mg—Zn, brass, etc.]), ceramics, and/or fiber reinforced materials (e.g., carbon fiber material, fiberglass, etc.). The protective and/or supportive skin of the medical device and/or one or more of the anchoring members can have the same or different flexibility, strength and/or rigidity.

In one non-limiting embodiment of the present invention, the anchoring members and the supportive skin (e.g., thin film, thin sheet, etc.) include one or more different materials. The one or more materials that are selected to form one or more portions of the medical device are typically selected to impart the desired properties on the medical device so that the medical device can 1) withstand the manufacturing process that is needed to produce the medical device (e.g., laser cutting, etching, MEMS (e.g., micro-machining, etc.) processes, masking processes, crimping, annealing, drawing, pilgering, electroplating, electro-polishing, chemical polishing, ion beam deposition or implantation, sputter coating, vacuum deposition, molding, melting, adhesive bonding, cutting, extruding, etching, heating, cooling, etc.); and 2) impart the desired properties to the medical device (e.g., strength, durability, biostability, biodegradability, bendability, radial strength, flexibility, tensile strength, biocompatibility, etc.). When one or more components of the medical device are formed by one or more polymers, the one or more polymers can be biostable, biodegradable, or bioabsorbable. The terms biodegradable or bioabsorbable are used interchangeably in this invention. Non-limiting examples of polymers that are considered to be biodegradable, bioresorbable, or bioerodable and which can be used to form one or more portions of the medical device include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g., DL-PLA), with and without additives (e.g., calcium phosphate glass), and/or other copolymers (e.g., poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that considered to be biostable and which can be used to form one or more portions of the medical device include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; polyparaphenylene terephthalamide (Kevlar®); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acryl ate, laurylacrylate, 2-hydroxy-propyl acrylate, polyhydroxyethyl, methacrylate/methylmethacrylate copolymers); glycosaminoglycans; alkyd resins; elastin; keratin; chitin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g., polystyrene); poly(vinyl ethers) (e.g., polyvinyl methyl ether); poly(vinyl ketones); poly(vinylidene halides) (e.g., polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g., polyvinyl chloride); polyacrylic acid; ethylene acrylic acid copolymer; ethylene vinyl acetate copolymer; polyvinyl alcohol; poly(hydroxyl alkylmethacrylate); Polyvinyl esters (e.g., polyvinyl acetate); and/or copolymers, blends, and/or composites of above. Non-limiting examples of polymers that can be made to be biodegradable and/or bioresorbable with modification and which can be used to form one or more portions of the medical device include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used.

In still another and/or alternative non-limiting aspect of the present invention, the medical device or one or more regions of the medical device can be at least partially formed by using microfabrication and/or micromachining technology used in creating Micro-Electro-Mechanical Systems (MEMS) such as, but not limited to, micro-machining, laser micro-machining, laser micro-machining, micro-molding, etc.; however, other or additional manufacturing techniques can be used. The medical device can include one or more surface structures (e.g., pore, channel, pit, rib, slot, notch, bump, teeth, well, hole, groove, etc.). These structures can be at least partially formed by MEMS (e.g., micro-machining, etc.) technology and/or other types of technology. The medical device can include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the medical device. For instance, one or more micro-structures can be positioned on one or more anchoring members, one or more cross structures, and/or the protective and/or supportive skin (e.g., thin film, thin sheet, etc.) of the medical device. Non-limiting examples of structures that can be formed on the medical device are illustrated in U.S. Pat. No. 6,974,475 and Publication Nos. 2004/0093076 and 2004/0093077, which are incorporated herein by reference. In one non-limiting embodiment of the invention, when one or more micro-structures are used on one or more anchoring members, the one or more micro-structures can be used to, but are not limited to, a) at least partially penetrate and/or at least partially secure to an inner wall surface of the body passageway to facilitate in the anchoring of the medical device to the body passageway, b) at least partially penetrate and/or at least partially secure to an inner wall surface of the body passageway to facilitate in local delivery of one or more chemical agents, c) at least partially provide structural mechanisms on the anchoring members to facilitate in the crimping and/or expansion of the anchoring members, and/or d) at least partially secure and/or at least partially connect one or more other components of the medical device to the anchoring members (e.g., cross members, protective and/or supportive skin, etc.). In another one non-limiting embodiment of the invention, when one or more micro-structures are used on one or more cross members, the one or more micro-structures can be used to, but are not limited to, a) at least partially penetrate and/or at least partially secure to an inner wall surface of the body passageway to facilitate in the anchoring of the medical device to the body passageway, b) at least partially penetrate and/or at least partially secure to an inner wall surface of the body passageway to facilitate in local delivery of one or more chemical agents, c) at least partially provide structural mechanisms on the cross members to facilitate in the crimping and/or expansion of the cross members, and/or d) at least partially secure and/or at least partially connect one or more other components of the medical device to the cross members (e.g., anchoring members, protective and/or supportive skin, etc.). In still another one non-limiting embodiment of the invention, when one or more micro-structures are used on the protective and/or supportive skin (e.g., thin film, thin sheet, etc.), the one or more micro-structures can be used to, but are not limited to, a) at least partially facilitate in the connecting and/or securing the protective and/or supportive skin (e.g., thin film, thin sheet, etc.) to a diseased area in the body passageway, b) at least partially facilitate in local delivery of one or more chemical agents to the diseased area, c) at least partially provide structural mechanisms on the protective and/or supportive skin (e.g., thin film, thin sheet, etc.) to facilitate in the crimping and/or expansion of the protective and/or supportive skin (e.g., thin film, thin sheet, etc.), and/or d) at least partially secure and/or at least partially connect one or more other components (e.g., anchoring members, cross members, etc.) of the medical device to the protective and/or supportive skin (e.g., thin film, thin sheet, etc.). Micro-structures, when formed to extend from one or more surface regions of the medical device, typically extend outwardly no more than about 1000 microns, and more typically less than about 600 microns, and more typically about 15-500 microns; however, other sizes can be used. A plurality of micro-structures can be clustered together or disbursed throughout the surface of the medical device. Similar shaped and/or sized micro-structures and/or surface structures can be used, or different shaped and/or sized micro-structures can be used on one or more portions of the medical device. When one or more surface structures and/or micro-structures are designed to extend from the surface of the medical device, the one or more surface structures and/or micro-structures can be formed in the extended position and/or be designed so as to extend from the medical device during and/or after deployment of the medical device in a treatment area. The micro-structures and/or surface structures can be designed to contain and/or be fluidly connected to a passageway, cavity, etc. in the medical device; however, this is not required. The one or more surface structures and/or micro-structures can be used to 1) at least partially facilitate in the expansion of one or more portions of the medical device (i.e., see non-limiting examples in U.S. Pat. No. 6,974,475 and Publication Nos. 2004/0093076 and 2004/0093077), 2) at least partially facilitate in maintaining the shape on one or more portions of the medical device in an expanded or unexpanded configuration (i.e., see non-limiting examples in U.S. Pat. No. 6,974,475 and Publication Nos. 2004/0093076 and 2004/0093077), 3) at least partially facilitate in anchoring the medical device at and/or about a treatment area, 4) at least partially facilitate in directing one or more chemical agents at or about a treatment area (e.g., vulnerable plaque, etc.), and/or 5) at least partially facilitate in connecting and/or securing one or more portions of the medical device together; however, this is not required. The one or more surface structures and/or micro-structures can be at least partially formed by MEMS (e.g., micro-machining, laser micro-machining, micro-molding, etc.) technology; however, this is not required. The one or more surface structures and/or micro-structures can be at least partially formed of a chemical agent and/or a polymer; however, this is not required. One or more of the surface structures and/or micro-structures can include one or more internal passageways that can include one or more materials (e.g., chemical agent, polymer, etc.); however, this is not required. One or more regions of the medical device, and/or one or more micro-structures and/or surface structures on the medical device can include a protective material that can be used to, but not limited to, 1) at least partially limit or prevent damage to one or more regions of the medical device when the medical device is a) packaged and/or stored, b) unpackaged, c) connected to and/or other secured and/or placed on another medical device, d) inserted into a treatment area, and/or e) handled by a user; 2) at least partially form a surface on the medical device to facilitate in the insertion of the medical device in a body passageway and/or through a delivery device (e.g., catheter, etc.); and/or 3) at least partially form a barrier between one or more micro-structures and/or surface structures and fluids in the body passageway so as to limit or prevent irritation (e.g., swelling, infection, etc.) of the body passageway by the medical device when the medical device is being inserted through the body passageway and/or is anchored in a region in the body passageway. The protective material, when used, can be 1) an at least partially biostable and/or at least partially biodegradable and/or 2) porous and/or non-porous. In non-limiting design, the protective material includes, but is not limited to, sugar (e.g., glucose, fructose, sucrose, etc.), carbohydrate compound, salt (e.g., NaCl, etc.), one or more polymers (e.g., parylene, PLGA, POE, PGA, PLLA, PAA, PEG, chitosan, etc.); however, other and/or additional materials can be used.

In yet another and/or alternative non-limiting aspect of the present invention, the one or more anchoring members on the medical device are expandable structures that have a first cross-sectional area which permits delivery of the anchoring member into a body passageway, and a second, expanded cross-sectional area. The expansion of one or more of the anchoring members of the medical device can be accomplished in a variety of manners. In one manner, one or more anchoring members are expanded to the second cross-sectional area by a radially, outwardly extending force applied at least partially from the interior region of the anchoring member (e.g., by use of a balloon, etc.). The one or more anchoring members can include heat sensitive materials (e.g., shape memory materials, etc.) that expand upon exposure to heat, thus not requiring a radially, outwardly extending force applied at least partially from the interior region of the anchoring member; however, such outwardly extending force can still be used with such a anchoring member. The second cross-sectional area of the anchoring member can be fixed or variable. The one or more anchoring members can have a first cross-sectional shape that is generally circular so as to form a substantially tubular portion of the medical device; however, the one or more anchoring members can have other cross-sectional shapes. The expansion of the one or more anchoring members can be accomplished by the bending of metal, by use of a shape memory material, by use of a biostable configuration and material, and/or by some mechanical expansion arrangement mechanism (e.g., see non-limiting examples of structures disclosed in U.S. Pat. No. 6,974,475 and Publication Nos. 2004/0093076 and 2004/0093077).

In still yet another and/or alternative non-limiting aspect of the present invention, the medical device can include one or more cross members that are connected to one or more anchoring members. The one or more cross members can be used to a) at least partially secure the two of more anchoring members together, and/or b) at least partially provide support to the protective and/or supportive skin (e.g., thin film, thin sheet, etc.) of the medical device. In one non-limiting embodiment of the present invention, the one or more cross members are designed to at least partially maintain one or more portions of the protective and/or supportive skin (e.g., thin film, thin sheet, etc.) on or in close proximity to the diseased area of the body passageway during and/or after one or more of the anchoring members are and/or have been expanded in the body passageway. The configuration and/or number of cross members on the medical device are non-limiting. In another and/or alternative non-limiting embodiment of the invention, one or more of the cross members have a shape similar to a rod or bar. In still another and/or alternative non-limiting embodiment of the invention, one or more of the cross members have a shape similar to a spiral. In yet another and/or alternative non-limiting embodiment of the invention, one or more of the cross members have a shape similar mesh design and/or a plurality of cross members can form the shape of a mesh design (i.e., a more solid structure having a plurality of holes, openings, slots, etc.). Such mesh designs can be similar to the side surfaces of stents such those illustrated in U.S. Pat. No. 6,206,916; U.S. Pat. No. 6,436,133; US 2004/0093076 and US 2004/0093077, and all the prior art cited in these patents and patent publications. In still another and/or alternative non-limiting embodiment of the invention, one or more of the cross members are at least partially secured to the peripheral edge of one or more anchoring members so that upon expansion of the one or more anchoring members, at least a portion of the one or more cross-members at least partially causes one or more portions of the protective and/or supportive skin (e.g., thin film, thin sheet, etc.) of the medical device to move toward and/or engage one or more portions of the diseased area on the body passageway. In yet another and/or alternative non-limiting embodiment of the invention, one or more of the cross members can extend completely about the other perimeter of the medical device or extend only a portion about the perimeter of the medical device. For instance, when the cross members are formed of rod or bar members, the cross members typically only extend about a portion of the outer perimeter of the medical device, thus forming only a portion of the outer peripheral surface of the medical device. When one or more cross members have and/or form a mesh-like configuration, the one or more cross members can extend completely or partially about the outer perimeter of the medical device. The one or more cross members can be formed integrally with the one or more anchoring members or be secured to the one or more anchoring members by one or more mechanisms (e.g., adhesive, melted bond, latch arrangement, clip arrangement, clamp arrangement, etc.). The one or more cross members can be made of a similar or a different material from the one or more anchoring members. One or more cross members can be formed from a material that undergoes plastic deformation when expanded (e.g., metal material, etc.), expanded by use of a shape memory material, expanded by use of a biostable configuration and material, and/or expanded by use of some mechanical expansion arrangement mechanism (e.g., see examples of non-limiting structures disclosed in U.S. Pat. No. 6,974,475 and Publication Nos. 2004/0093076 and 2004/0093077). The one or more cross members can be formed by one or more micro-machining techniques; however, this is not required.

In a further and/or alternative non-limiting aspect of the present invention, the protective and/or supportive skin (e.g., thin film, thin sheet, etc.) on the medical device can include one or more structural members that can be use to create a shape for one or more regions of the protective and/or supportive skin and/or provide structural rigidity to one or more regions of the protective and/or supportive skin when the one or more anchoring members have been expanded. These one or more structural members can be used in conjunction with one or more cross members on the medical device or can be used as a substitution of one or more or all of the cross members. In one non-limiting embodiment of the invention, one or more of the structural members have a curved or spiral configuration. As can be appreciated, many other or additional configurations of these structural members can be used. The one or more structural members can be formed integrally with the protective and/or supportive skin and/or be secured to the protective and/or supportive skin by one or more mechanisms (e.g., adhesive, melted bond, latch arrangement, clip arrangement, clamp arrangement, etc.). The one or more structural members can be made of a similar or a different material from the protective and/or supportive skin. One or more structural members can be formed from a material than can be bent when expanded (e.g., metal material, etc.), expanded by use of a shape memory material, expanded by use of a biostable configuration and material, and/or expanded by use of some mechanical expansion arrangement mechanism (e.g., see examples of non-limiting structures disclosed in U.S. Pat. No. 6,974,475 and Publication Nos. 2004/0093076 and 2004/0093077). The one or more structural members can be formed by one or more micro-machining techniques; however, this is not required.

In still a further and/or alternative non-limiting aspect of the present invention, the medical device can include one or more chemical agents to facilitate in the success of the medical device and/or treated area. One or more regions of the medical device (i.e., anchoring member, protective and/or supportive skin, cross member, structural member, micro-structure, surface structure, etc.) can include, contain and/or be coated with one or more chemical agents. The term chemical agent includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more chemical agents include, but are not limited to viral, fungus and/or bacteria infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. Non-limiting examples of chemical agents that can be used include, but are not limited to, 5-Fluorouracil and/or derivatives thereof; 5-Phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; chemical agents that modulate intracellular Ca2+ transport such as L-type (e.g., diltiazem, nifedipine, verapamil, etc.) or T-type Ca2+ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; anti-biotics; anti-coagulant compounds and/or derivatives thereof; anti-fibrosis compounds and/or derivatives thereof; antifungal compounds and/or derivatives thereof; anti-inflammatory compounds and/or derivatives thereof; Anti-Invasive Factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; an platelet compounds and/or derivatives thereof; anti-proliferative and/or derivatives thereof; anti-thrombogenic agents and/or derivatives thereof; argatroban and/or derivatives thereof; ap-1 inhibitors and/or derivatives thereof (e.g., for tyrosine kinase, protein kinase C, myosin light chain kinase, Ca2+/calmodulin kinase II, casein kinase II, etc.); aspirin and/or derivatives thereof; azathioprine and/or derivatives thereof; $-Estradiol and/or derivatives thereof; $−1-anticollagenase and/or derivatives thereof; calcium channel blockers and/or derivatives thereof; calmodulin antagonists and/or derivatives thereof (e.g., H7, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof; endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP3 receptor and/or derivatives thereof; inhibitors of the sodium/hydrogen antiporter and/or derivatives thereof (e.g., amiloride and derivatives thereof, etc.); insulin and/or derivatives thereof; Interferon alpha 2 Macroglobulin and/or derivatives thereof; ketoconazole and/or derivatives thereof; Lepirudin and/or derivatives thereof; LISINOPRIL and/or derivatives thereof; LOVASTATIN and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; Plasminogen Activator Inhibitor-1 and/or derivatives thereof; Plasminogen Activator Inhibitor-2 and/or derivatives thereof; Platelet Factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H3P32O4, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof; TH1 and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; Tissue Inhibitor of Metalloproteinase-1 and/or derivatives thereof; Tissue Inhibitor of Metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof. As can be appreciated, the chemical agent can include one or more derivatives of the above listed compounds and/or other compounds. In one non-limiting embodiment, the chemical agent includes, but is not limited to, trapidil, Trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, 5-Phenylmethimazole, 5-Phenylmethimazole derivatives, GM-CSF (granulo-cytemacrophage colony-stimulating-factor), GM-CSF derivatives, statins or HMG-CoA reductase inhibitors forming a class of hypolipidemic agents, combinations, or analogs thereof, or combinations thereof. The type and/or amount of chemical agent included in the device and/or coated on the device can vary. When two or more chemical agents are included in and/or coated on the device, the amount of two or more chemical agents can be the same or different. The type and/or amount of chemical agent included on, in and/or in conjunction with the device are generally selected to address one or more clinical events. Typically the amount of chemical agent included on, in and/or used in conjunction with the device is about 0.01-100 ug per mm² and/or at least about 0.01 weight percent of device; however, other amounts can be used. In one non-limiting embodiment of the invention, the device can be partially of fully coated and/or impregnated with one or more chemical agents to facilitate in the success of a particular medical procedure. The amount of two of more chemical agents on, in and/or used in conjunction with the device can be the same or different. The one or more chemical agents can be coated on and/or impregnated in the device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the invention, the type and/or amount of chemical agent included on, in and/or in conjunction with the device is generally selected for the treatment of one or more clinical events. Typically the amount of chemical agent included on, in and/or used in conjunction with the device is about 0.01-100 ug per mm² and/or at least about 0.01-100 weight percent of the device; however, other amounts can be used. The amount of two of more chemical agents on, in and/or used in conjunction with the device can be the same or different. For instance, portions of the device to provide local and/or systemic delivery of one or more chemical agents in and/or to a body passageway to a) inhibit or prevent thrombosis, in-stent restenosis, vascular narrowing and/or restenosis after the device has been inserted in an(/or connected to a body passageway, b) at least partially passivate, remove, encapsulate, and/or dissolve lipids, fibroblast, fibrin, etc. in a body passageway so as to at least partially remove such materials and/or to passivate such vulnerable materials (e.g., vulnerable plaque, etc.) in the body passageway in the region of the device and/or downstream of the device. As can be appreciated, the one or more chemical agents can have many other or additional uses. In still another and/or alternative non-limiting example, the device is coated with and/or includes one or more chemical agents such as, but not limited to chemical agents associated with thrombolytics, vasodilators, anti-hypertensive agents, antimicrobial or anti-biotic, anti-mitotic, anti-proliferative, anti-secretory agents, non-steroidal anti-inflammatory drugs, immunosuppressive agents, growth factors and growth factor antagonists, endothelial growth factors and growth factor antagonists, antitumor and/or chemotherapeutic agents, anti-polymerases, anti-viral agents, anti-body targeted therapy agents, hormones, anti-oxidants, biologic components, radio-therapeutic agents, radiopaque agents and/or radio-labeled agents. In addition to these chemical agents, the device can be coated with and/or include one or more chemical agents that are capable of inhibiting or preventing any adverse biological response by and/or to the device that could possibly lead to device failure and/or an adverse reaction by human or animal tissue. A wide range of chemical agents thus can be used.

In a further and/or alternative non-limiting aspect of the present invention, the one or more chemical agents on and/or in the device, when used on the device, can be released in a controlled manner so the area in question to be treated is provided with the desired dosage of chemical agent over a sustained period of time. As can be appreciated, controlled release of one or more chemical agents on the device is not always required and/or desirable. As such, one or more of the chemical agents on and/or in the device can be uncontrollably released from the device during and/or after insertion of the device in the treatment area. It can also be appreciated that one or more chemical agents on and/or in the device can be controllably released from the device and one or more chemical agents on and/or in the device can be uncontrollably released from the device. It can also be appreciated that one or more chemical agents on and/or in one region of the device can be controllably released from the device and one or more chemical agents on and/or in the device can be uncontrollably released from another region on the device. As such, the device can be designed such that 1) all the chemical agent on and/or in the device is controllably released, 2) some of the chemical agent on and/or in the device is controllably released and some of the chemical agent on the device is non-controllably released, or 3) none of the chemical agent on and/or in the device is controllably released. The device can also be designed such that the rate of release of the one or more chemical agents from the device is the same or different. The device can also be designed such that the rate of release of the one or more chemical agents from one or more regions on the device is the same or different. Non-limiting arrangements that can be used to control the release of one or more chemical agent from the device include a) at least partially coat one or more chemical agents with one or more polymers, b) at least partially incorporate and/or at least partially encapsulate one or more chemical agents into and/or with one or more polymers, c) insert one or more chemical agents in pores, passageway, cavities, etc. in the device and at least partially coat or cover such pores, passageway, cavities, etc. with one or more polymers, and/or incorporate one or more chemical agents in the one or more polymers that at least partially form the device. As can be appreciated, other or additional arrangements can be used to control the release of one or more chemical agent from the device. The one or more polymers used to at least partially control the release of one or more chemical agent from the device can be porous or non-porous. The one or more chemical agents can be inserted into and/or applied to one or more surface structures and/or micro-structures on the device, and/or be used to at least partially form one or more surface structures and/or micro-structures on the device. As such, the one or more chemical agents on the device can be 1) coated on one or more surface regions of the device, 2) inserted and/or impregnated in one or more surface structures and/or micro-structures, etc. of the device, and/or 3) form at least a portion or be included in at least a portion of the structure of the device. When the one or more chemical agents are coated on the device, the one or more chemical agents can, but is not required to, 1) be directly coated on one or more surfaces of the device, 2) be mixed with one or more coating polymers or other coating materials and then at least partially coated on one or more surfaces of the device, 3) be at least partially coated on the surface of another coating material that has been at least partially coated on the device, and/or 4) be at least partially encapsulated between a) a surface or region of the device and one or more other coating materials and/or b) two or more other coating materials. As can be appreciated, many other coating arrangements can be additionally or alternatively used. When the one or more chemical agents are inserted and/or impregnated in one or more portions of the device, one or more surface structure and/or micro-structures of the device, and/or one or more surface structures and/or micro-structures of the device, 1) one or more other polymers can be applied at least partially over the one or more surface structure and/or micro-structures, surface structures and/or micro-structures of the device, 2) one or more polymers can be combined with one or more chemical agents, and/or 3) one or more polymers can be coated over or more portions of the body of the device; however, this is not required. As such, the one or more chemical agents can be 1) embedded in the structure of the device; 2) positioned in one or more surface structure and/or micro-structures of the device; 3) encapsulated between two polymer coatings; 4) encapsulated between the base structure and a polymer coating; 5) mixed in the base structure of the device that includes at least one polymer coating; or 6) one or more combinations of 1, 2, 3, 4 and/or 5. In addition or alternatively, the one or more coatings of the one or more polymers on the device can include 1) one or more coatings of non-porous polymers; 2) one or more coatings of a combination of one or more porous polymers and one or more non-porous polymers; 3) one or more coating of porous polymer, or 4) one or more combinations of options 1, 2, and 3. As can be appreciated different chemical agents can be located in and/or between different polymer coating layers and/or on and/or the structure of the device. As can also be appreciated, many other and/or additional coating combinations and/or configurations can be used. In a further and/or alternative non-limiting embodiment of the present invention, the device can be embedded with and/or impregnated with one or more chemical agents using a solvent to temporarily and/or permanently increase the porosity of the structure of a non-porous and/or porous polymer coating and/or device and be used to transport one or more chemical agents into the matrix of the device. One or more solvents can be used to transport one or more chemical agents. Solvent suitability is a function of compatibility with one or more chemical agents and one or more materials of the device. Non-limiting examples of solvents include Dimethyl sulfoxide (DMSO), chloroform, ethylene, methanol, ethyl acetate, and the broader class of biocompatible or non-biocompatible solvents. The concentration of one or more chemical agents, the type of polymer, the type and/or shape of surface structure and/or micro-structures in the device and/or the coating thickness of one or more chemical agents can be used to control the release time, the release rate and/or the dosage amount of one or more chemical agents; however, other or additional combinations can be used. As such, the chemical agent and polymer system combination and location on the device can be numerous. As can also be appreciated, one or more chemical agents can be deposited on the top surface of the device to provide an initial uncontrolled burst effect of the one or more chemical agents prior to 1) the control release of the one or more chemical agents through one or more layers of polymer system that include one or more nonporous polymers and/or 2) the uncontrolled release of the one or more chemical agents through one or more layers of polymer system. The one or more chemical agents and/or polymers can be coated on and/or impregnated in the device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. The thickness of each polymer layer and/or layer of chemical agent is generally at least about 0.01 μm and is generally less than about 150 μm. In one non-limiting embodiment, the thickness of a polymer layer and/or layer of chemical agent is about 0.02-75 μm, more particularly about 0.05-50 μm, and even more particularly about 1-30 μm. When the device includes and/or is coated with one or more chemical agents such that at least one of the chemical agents is at least partially controllably released from the device, the need or use of body-wide therapy for extended periods of time can be reduced or eliminated. In the past, the use of body-wide therapy was used by the patient long after the patient left the hospital or other type of medical facility. This body-wide therapy could last days, weeks, months or sometimes over a year after surgery. The device of the present invention can be applied or inserted into a treatment area and 1) merely requires reduced use and/or extended use of systemic therapy after application or insertion of the device or 2) does not require use and/or extended use of systemic therapy after application or insertion of the device. As can be appreciated, use and/or extended use of systemic therapy can be used after application or insertion of the device at the treatment area. In one non-limiting example, no body-wide therapy is needed after the insertion of the device into a patient. In another and/or alternative non-limiting example, short term use of systemic therapy is needed or used after the insertion of the device into a patient. Such short term use can be terminated after the release of the patient from the hospital or other type of medical facility, or one to two days or weeks after the release of the patient from the hospital or other type of medical facility; however, it will be appreciated that other time periods of systemic therapy can be used. As a result of the use of the device of the present invention, the use of systemic therapy after a medical procedure involving the insertion of a device into a treatment area can be significantly reduced or eliminated.

In another and/or alternative non-limiting aspect of the present invention, the device, when including and/or is coated with one or more chemical agents, can include and/or can be coated with one or more chemical agents that are the same or different in different regions of the device and/or have differing amounts and/or concentrations in differing regions of the device. For instance, the device can a) be coated with and/or include one or more chemical agents on at least one portion of the device and at least another portion of the device is not coated with and/or includes chemical agent; b) be coated with and/or include one or more chemical agents on at least one portion of the device that is different from one or more chemical agents on at least another portion of the device; c) be coated with and/or include one or more chemical agents at a concentration on at least one portion of the device that is different from the concentration of one or more chemical agents on at least another portion of the device; etc.

In still another and/or alternative non-limiting aspect of the present invention, one or more surfaces of the device can be treated to achieve the desired coating properties of the one or more chemical agents and one or more polymers coated on and/or incorporated in the device. Such surface treatment techniques include, but are not limited to, cleaning, buffing, smoothing, etching (chemical etching, plasma etching, etc.) achieved through a variety of techniques. When an etching process is used, various gasses can be used for such a surface treatment process such as, but not limited to, carbon dioxide, nitrogen, oxygen, Freon, helium, hydrogen, etc. The plasma etching process can be used to clean the surface of the device, change the surface properties of the device so as to affect the adhesion properties, lubricity properties, etc. of the surface of the device. As can be appreciated, other or additional surface treatment processes can be used prior to and/or after the coating of one or more chemical agents and/or polymers on the surface of the device. In one non-limiting manufacturing process, one or more portions of the device are cleaned and/or plasma etched; however, this is not required. Plasma etching can be used to clean the surface of the device, and/or to form one or more non-smooth surfaces on the device to facilitate in the adhesion of one or more coatings of chemical agents and/or one or more coatings of polymer on the device. Once one or more surface regions of the device have been treated, one or more coatings of polymer and/or chemical agent can be applied to one or more regions of the device. For instance, 1) one or more layers of porous or non-porous polymer can be coated on an outer and/or inner surface of the device, 2) one or more layers of chemical agent can be coated on an outer and/or interior surface of the device, or 3) one or more layers of porous or non-porous polymer that includes one or more chemical agents can be coated on an outer and/or interior surface of the device. The one or more layers of chemical agent can be applied to the device by a variety of coating techniques (e.g., dipping, rolling, brushing, spraying, particle atomization, etc.). One non-limiting coating technique is by an ultrasonic mist coating process wherein ultrasonic waves are used to break up the droplet of chemical agent and form a mist of very fine droplets. These fine droplets have an average droplet diameter of about 0.1-3 microns. The fine droplet mist facilitates in the formation of a uniform coating thickness and can increase the coverage area on the device.

In a further and/or alternative non-limiting aspect of the present invention, the device or one or more regions of the device can be constructed by use of one or more microfabrication and/or micromachining technology used in creating Micro-Electro-Mechanical Systems (MEMS, e.g., micro-machining, laser micro machining, micro-molding, etc.); however, other or additional manufacturing techniques can be used. The device can include one or more surface structures (e.g., pore, channel, pit, rib, slot, notch, bump, teeth, well, hole, groove, etc.). These structures can be at least partially formed by MEMS technology and/or other types of technology. The device can include one or more micro-structures (e.g., micro-needle, micro-pore, micro-cylinder, micro-cone, micro-pyramid, micro-tube, microparallelopiped, micro-prism, micro-hemisphere, teeth, rib, ridge, ratchet, hinge, zipper, zip-tie like structure, etc.) on the inner, outer, or edge surface of the device. Non-limiting examples of structures that can be formed on the devices such as stent, graft, and/or other suitable devices are illustrated in United States Patent Publication Nos. 2004/0093076 and 2004/0093077, which are incorporated herein by reference. Typically, the micro-structures, when formed, extend from or into the outer surface no more than about 1000 microns, and more typically less than about 1000 microns; however, other sizes can be used. The micro-structures can be clustered together or disbursed throughout the surface of the device. Similar shaped and/or sized micro-structures and/or surface structures can be used, or different shaped and/or sized microstructures can be used. When one or more surface structures and/or micro-structures are designed to extend from the outer and/or inner surface of the device, the one or more surface structures and/or micro-structures can be formed in the extended position and/or be designed so as to extend from the device during and/or after deployment of the device in a treatment area. The micro-structures and/or surface structures can be designed to contain one or more chemical agents and/or be connected to a passageway, cavity, etc. containing one or more chemical agents; however, this is not required. The one or more surface structures and/or micro-structures can be used to engage and/or penetrate surrounding tissue or organs once the device has been positioned on and/or in a patient; however, this is not required. In another further and/or alternative non-limiting aspect of the present invention, the micro-structures and/or surface structures can be design to modify surface friction between the device and/or additional devices. For example, micro-structures and/or surface structures created on the inner surface of the device may be used to increase retention of a stent, graft, and/or other suitable device on a delivery catheter. In another further and/or alternative non-limiting aspect of the present invention, the micro-structures and/or surface structures can be design to create a system of undulations and/or crevasses used to facilitate growth of tissue. In one non-limiting aspect, the micro-structures and/or surface structures can be created on a film that could further be rolled into a shunt for neural regeneration, where the micro-structures and/or surface structures can provide a lattice to support and/or facilitate nerve growth. The one or more surface structures and/or micro-structures can be used to facilitate in forming or maintaining a shape of a device (i.e., see devices in United States Patent Publication Nos. 2004/0093076 and 2004/0093077). The one or more surface structures and/or micro-structures can be at least partially formed by MEMS technology; however, this is not required. In one non-limiting embodiment, the one or more surface structures and/or microstructures can be at least partially formed of a chemical agent, polymer, chemical agent-polymer mixture, and/or layering of polymer and chemical agent. One or more of the surface structures and/or micro-structures can include one or more internal passageways that can include one or more materials (e.g., chemical agent, polymer, etc.); however, this is not required. In another further and/or alternative non-limiting aspect of the present invention, one or more internal passageways can be either connected and/or separated in part. The one or more surface structures and/or micro-structures can be formed by a variety of processes (e.g., machining, chemical modifications, chemical reactions, MEMS technology, etching, laser cutting, etc.). The one or more coatings and/or one or more surface structures and/or micro-structures of the device can be used for a variety of purposes such as, but not limited to, 1) increasing the bonding and/or adhesion of one or more chemical agents, adhesives, marker materials and/or polymers to the device, 2) changing the appearance or surface characteristics of the device, and/or 3) controlling the release rate of one or more chemical agents. The one or more microstructures and/or surface structures can be biostable, biodegradable, etc. One or more regions of the device that are at least partially formed by MEMS technology can be biostable, biodegradable, etc. The device or one or more regions of the device can be at least partially covered and/or filled with a protective material so as to at least partially protect one or more regions of the device, and/or one or more microstructures and/or surface structures on the device from damage. One or more regions of the device, and/or one or more micro-structures and/or surface structures on the device can be damaged when the device is 1) packaged and/or stored, 2) unpackaged, 3) connected to and/or otherwise secured and/or placed on another device, 4) inserted into a treatment area, 5) handled by a user, and/or 6) form a barrier between one or more micro-structures and/or surface structures and fluids in the body passageway. As can be appreciated, the device can be damaged in other or additional ways. The protective material can be used to protect the device and one or more micro-structures and/or surface structures from such damage. The protective material can include one or more polymers previously identified above. The protective material can be 1) biostable and/or biodegradable and/or 2) porous and/or non-porous. In one non-limiting design, the polymer is at least partially biodegradable so as to at least partially expose one or more micro-structure and/or surface structure to the environment after the device has been at least partially inserted into a treatment area. In another and/or additional non-limiting design, the protective material includes, but is not limited to, sugar (e.g., glucose, fructose, sucrose, etc.), carbohydrate compound, salt (e.g., NaCl, etc.), parylene, PLGA, POE, PGA, PLLA, PAA, PEG, chitosan and/or copolymers, blends, and/or composites of above and/or derivatives of one or more of these polymers; however, other and/or additional materials can be used. In still another and/or additional non-limiting design, the thickness of the protective material is generally less than about 300 microns, and typically less than about 150 microns; however, other thicknesses can be used depending upon the material chose of the protective material. The protective material can be coated by one or more mechanisms previously described herein.

In still yet another and/or alternative non-limiting aspect of the present invention, the device can include and/or be used with a physical hindrance. The physical hindrance can include, but is not limited to, an adhesive, a sheath, a magnet, tape, wire, string, etc. The physical hindrance can be used to 1) physically retain one or more regions of the device in a particular form or profile, 2) physically retain the device on a particular deployment device, 3) protect one or more surface structures and/or micro-structures on the device, and/or 4) form a barrier between one or more surface regions, surface structures and/or microstructures on the device and the fluids in a body passageway. As can be appreciated, the physical hindrance can have other and/or additional functions. The physical hindrance is typically a biodegradable material; however, a biostable material can be used. The physical hindrance can be designed to withstand sterilization of the device; however, this is not required. The physical hindrance can be applied to, included in and/or be used in conjunction with one or more devices. Additionally or alternatively, the physical hindrance can be designed to be used with and/or in conjunction with a device for a limited period of time and then 1) disengage from the device after the device has been partially or fully deployed and/or 2) dissolve and/or degrade during and/or after the device has been partially or fully deployed; however, this is not required. Additionally or alternatively, the physical hindrance can be designed and be formulated to be temporarily used with a device to facilitate in the deployment of the device; however, this is not required. In one non-limiting use of the physical hindrance, the physical hindrance is designed or formulated to at least partially secure a device to another device that is used to at least partially transport the device to a location for treatment. In another and/or alternative non-limiting use of the physical hindrance, the physical hindrance is designed or formulated to at least partially maintain the device in a particular shape or form until the device is at least partially positioned in a treatment location. In still another and/or alternative non-limiting use of the physical hindrance, the physical hindrance is designed or formulated to at least partially maintain and/or secure one type of device to another type of medical instrument or device until the device is at least partially positioned in a treatment location. The physical hindrance can also or alternatively be designed and formulated to be used with a device to facilitate in the use of the device. In one non-limiting use of the physical hindrance, when in the form of an adhesive, can be formulated to at least partially secure a device to a treatment area so as to facilitate in maintaining the device at the treatment area. For instance, the physical hindrance can be used in such use to facilitate in maintaining a device on or at a treatment area until the device is properly secured to the treatment area by sutures, stitches, screws, nails, rod, etc.; however, this is not required. Additionally or alternatively, the physical hindrance can be used to facilitate in maintaining a device on or at a treatment area until the device has partially or fully accomplished its objective. The physical hindrance is typically a biocompatible material so as to not cause unanticipated adverse effects when properly used. The physical hindrance can be biostable or biodegradable (e.g., degrades and/or is absorbed, etc.). When the physical hindrance includes or is one or more adhesives, the one or more adhesives can be applied to the device by, but is not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition on the device. The physical hindrance can also or alternatively form at least a part of the device. One or more regions and/or surfaces of a device can also or alternatively include the physical hindrance. The physical hindrance can include one or more chemical agents and/or other materials (e.g., marker material, polymer, etc.); however, this is not required. When the physical hindrance is or includes an adhesive, the adhesive can be formulated to controllably release one or more chemical agents in the adhesive and/or coated on and/or contained within the device; however, this is not required. The adhesive can also or alternatively control the release of one or more chemical agents located on and/or contained in the device by forming a penetrable or non-penetrable barrier to such chemical agents; however, this is not required. The adhesive can include and/or be mixed with one or more polymers; however, this is not required. The one or more polymers can be used to 1) control the time of adhesion provided by said adhesive, 2) control the rate of degradation of the adhesive, and/or 3) control the rate of release of one or more chemical agents from the adhesive and/or diffusing or penetrating through the adhesive layer; however, this is not required. When the physical hindrance includes a sheath, the sheath can be designed to partially or fully encircle the device. The sheath can be designed to be physically removed from the device after the device is deployed to a treatment area; however, this is not required. The sheath can be formed of a biodegradable material that at least partially degrades over time to at least partially expose one or more surface regions, micro-structures and/or surface structures of the device; however, this is not required. The sheath can include and/or be at least partially coated with one or more chemical agents. The sheath includes one or more polymers; however, this is not required. The one or more polymers can be used for a variety of reasons such as, but not limited to, 1) forming a portion of the sheath, 2) improving a physical property of the sheath (e.g., improve strength, improve durability, improve biocompatibility, reduce friction, etc.), and/or 3 at least partially controlling a release rate of one or more chemical agents from the sheath. As can be appreciated, the one or more polymers can have other or additional uses on the sheath.

In still another and/or alternative non-limiting aspect of the invention, the device can be used in conjunction with one or more other chemical agents that are not on the device. For instance, the success of the device can be improved by infusing, injecting or consuming orally one or more chemical agents. Such chemical agents can be the same and/or different from the one or more chemical agents on and/or in the device. Such use of one or more chemical agents are commonly used in systemic treatment of a patient after a medical procedure such as systemic therapy after the device has been inserted in the treatment area can be reduced or eliminated by use of the novel alloy. Although the device of the present invention can be designed to reduce or eliminate the need for long periods of systemic therapy after the device has been inserted in the treatment area, the use of one or more chemical agents can be used in conjunction with the device to enhance the success of the device and/or reduce or prevent the occurrence of in-stent restenosis, vascular narrowing, and/or thrombosis and/or promote tissue growth (e.g., endothelium and/or neural tissue). For instance, solid dosage forms of chemical agents for oral administration, and/or for other types of administration (e.g., suppositories, etc.) can be used. Such solid forms can include, but are not limited to, capsules, tablets, effervescent tablets, chewable tablets, pills, powders, sachets, granules and gels. The solid form of the capsules, tablets, effervescent tablets, chewable tablets, pills, etc. can have a variety of shapes such as, but not limited to, spherical, cubical, cylindrical, pyramidal, and the like. In such solid dosage form, one or more chemical agents can be admixed with at least one filler material such as, but not limited to, sucrose, lactose or starch; however, this is not required. Such dosage forms can include additional substances such as, but not limited to, inert diluents (e.g., lubricating agents, etc.). When capsules, tablets, effervescent tablets or pills are used, the dosage form can also include buffering chemical agents; however, this is not required. Soft gelatin capsules can be prepared to contain a mixture of the one or more chemical agents in combination with vegetable oil or other types of oil; however, this is not required. Hard gelatin capsules can contain granules of the one or more chemical agents in combination with a solid carrier such as, but not limited to, lactose, potato starch, corn starch, cellulose derivatives of gelatin, etc; however, this is not required. Tablets and pills can be prepared with enteric coatings for additional time release characteristics; however, this is not required. Liquid dosage forms of the one or more chemical agents for oral administration can include pharmaceutically acceptable emulsions, solutions, suspensions, syrups, elixirs, etc.; however, this is not required. In one non-limiting embodiment, when at least a portion of one or more chemical agents is inserted into a treatment area (e.g., gel form, paste form, etc.) and/or provided orally (e.g., pill, capsule, etc.) and/or anally (suppository, etc.), one or more of the chemical agents can be controllably released; however, this is not required. In one non-limiting example, one or more chemical agents can be given to a patient in solid dosage form and one or more of such chemical agents can be controllably released from such solid dosage forms. In another and/or alternative non-limiting example trapidil, trapidil derivatives, taxol, taxol derivatives, cytochalasin, cytochalasin derivatives, paclitaxel, paclitaxel derivatives, rapamycin, rapamycin derivatives, 5-Phenylmethimazole, 5-Phenylmethimazole derivatives, GM-CSF, GM-CSF derivatives, or analogs, or combinations thereof are given to a patient prior to, during and/or after the insertion of the device in a treatment area. Certain types of chemical agents may be desirable to be present in a treated area for an extended period of time in order to utilize the full or nearly full clinical potential of the chemical agent. For instance, Trapidil and/or trapidil derivatives is a compound that has many clinical attributes including, but not limited to, anti-platelet effects, inhibition of smooth muscle cells and monocytes, fibroblast proliferation and increased MAPK-1 which in turn deactivates kinase, a vasodilator, etc. These attributes can be effective in improving the success of a device that has been inserted at a treatment area. In some situations, these positive effects of trapidil and/or Trapidil derivatives need to be prolonged in a treatment area in order to achieve complete clinical competency. Trapidil and/or trapidil derivatives has a half life in vivo of about 2-4 hours with hepatic clearance of 48 hours. In order to utilize the full clinical potential of trapidil and/or trapidil derivatives, trapidil and/or trapidil derivatives should be metabolized over an extended period of time without interruption; however, this is not required. By inserting trapidil and/or trapidil derivatives in a solid dosage form, the trapidil and/or trapidil derivatives could be released in a patient over extended periods of time in a controlled manner to achieve complete or nearly complete clinical competency of the trapidil and/or trapidil derivatives. In another and/or alternative non-limiting example, one or more chemical agents are at least partially encapsulated in one or more polymers. The one or more polymers can be biodegradable, non-biodegradable, porous, and/or non-porous. When the one or more polymers are biodegradable, the rate of degradation of the one or more biodegradable polymers can be used to at least partially control the rate at which one or more chemical agents that are released into a body passageway and/or other parts of the body over time. The one or more chemical agents can be at least partially encapsulated with different polymer coating thicknesses, different numbers of coating layers, and/or with different polymers to alter the rate at which one or more chemical agents are released in a body passageway and/or other parts of the body over time. The rate of degradation of the polymer is principally a function of 1) the water permeability and solubility of the polymer, 2) chemical composition of the polymer and/or chemical agent, 3) mechanism of hydrolysis of the polymer, 4) the chemical agent encapsulated in the polymer, 5) the size, shape and surface volume of the polymer, 6) porosity of the polymer, 7) the molecular weight of the polymer, 8) the degree of cross-linking in the polymer, 9) the degree of chemical bonding between the polymer and chemical agent, and/or 10) the structure of the polymer and/or chemical agent. As can be appreciated, other factors may also affect the rate of degradation of the polymer. When the one or more polymers are biostable, the rate at when the one or more chemical agents are released from the biostable polymer is a function of 1) the porosity of the polymer, 2) the molecular diffusion rate of the chemical agent through the polymer, 3) the degree of cross-linking in the polymer, 4) the degree of chemical bonding between the polymer and chemical agent, 5) chemical composition of the polymer and/or chemical agent, 6) the chemical agent encapsulated in the polymer, 7) the size, shape and surface volume of the polymer, and/or 8) the structure of the polymer and/or chemical agent. As can be appreciated, other factors may also affect the rate of release of the one or more chemical agents from the biostable polymer. Many different polymers can be used such as, but not limited to, aliphatic polyester compounds (e.g., PLA (i.e., poly(D, L-lactic acid), poly(L-lactic acid)), PLGA (i.e., poly(lactide-co-glycoside), etc.), POE, PEG, PLLA, parylene, chitosan and/or copolymers, blends, and/or composites of above and/or derivatives of one or more of these polymers. As can be appreciated, the at least partially encapsulated chemical agent can be introduced into a patient by means other than by oral introduction, such as, but not limited to, injection, topical applications, intravenously, eye drops, nasal spray, surgical insertion, suppositories, intrarticularly, intraocularly, intranasally, intradermally, sublingually, intravesically, intrathecally, intraperitoneally, intracranially, intramuscularly, subcutaneously, directly at a particular site, and the like.

In still yet a further and/or alternative non-limiting aspect of the invention, the medical device can include a marker material that facilitates enabling the medical device to be properly positioned in a body passageway. The marker material is typically designed to be visible to electromagnetic waves (e.g., x-rays, microwaves, visible light, inferred waves, ultraviolet waves, etc.); sound waves (e.g., ultrasound waves, etc.); magnetic waves (e.g., MRI, etc.); and/or other types of electromagnetic waves (e.g., microwaves, visible light, inferred waves, ultraviolet waves, etc.). In one non-limiting embodiment, the marker material is visible to x-rays (i.e., radiopaque). The marker material can form all or a portion of the medical device and/or be coated on one or more portions of the medical device (i.e., anchoring member, protective and/or supportive skin, cross member, structural member, micro-structure, surface structure, etc.). The location of the marker material can be on one or multiple locations on the medical device. The size of the one or more regions that include the marker material can be the same or different. The marker material can be spaced at defined distances from one another so as to form ruler-like markings on the medical device to facilitate in the positioning of the medical device in a body passageway; however, this is not required. In one non-limiting embodiment, a marker material is positioned on at least one of the anchoring members to mark at least one of the medical device. In one non-limiting aspect of this embodiment, a marker material is positioned on each anchoring member of the medical device so as to mark each end of the medical device. In another and/or alternative non-limiting aspect of this embodiment, a marker material is positioned on one or more cross members and/or structural members so as to mark the region between two of more anchoring members and/or to at least partially mark the location of the protective and/or supportive skin. The marker material can include a rigid and/or flexible material. The marker material can be a biostable or biodegradable material. When the marker material is a rigid material, the marker material is typically formed of a metal material (e.g., metal band, metal plating, etc.); however, other or additional materials can be used. When the marker material is a flexible material, the marker material typically is formed of one or more polymers that are marker materials in-of-themselves and/or include one or more metal powders and/or metal compounds; however, other or additional materials can be used. In still another and/or alternative non-limiting embodiment, at least one marker is flexible and includes one or more metal powders in combinations with parylene, PLGA, POE, PGA, PLLA, PAA, PEG, chitosan and/or derivatives of one or more of these polymers. In yet another and/or alternative non-limiting embodiment, at least one marker is a flexible marker material and includes one or more metals and/or metal powders of aluminum, barium, bismuth, cobalt, copper, chromium, gold, iron, stainless steel, titanium, vanadium, nickel, zirconium, niobium, lead, molybdenum, platinum, yttrium, calcium, rare earth metals, rhenium, zinc, silver, depleted radioactive elements, tantalum and/or tungsten; and/or compounds thereof. The marker material can be coated with a polymer protective material; however, this is not required. When the marker material is coated with a polymer protective material, the polymer coating can be used to, but not limited to, 1) at least partially insulate the marker material from body fluids, 2) facilitate in retaining the marker material on the medical device, 3) at least partially shield the marker material from damage during a medical procedure and/or 4) provide a desired surface profile on the medical device. As can be appreciated, the polymer coating can have other or additional uses. The polymer protective coating, when used, can be a biostable polymer or a biodegradable polymer (e.g., degrades and/or is absorbed). The coating thickness of the protective coating polymer material, when used, is typically less than about 300 microns; however, other thickness can be used. In one non-limiting aspect of this embodiment, the protective coating material ,when used, includes parylene, PLGA, POE, PGA, PLLA, PAA, PEG, chitosan and/or derivatives of one or more of these polymers.

In another and/or alternative non-limiting aspect of the invention, the medical device can include an adhesion agent to facilitate in securing of one or more portions of the medical device to a diseased area of a body passageway and/or the region about the diseased area of a body passageway. The adhesion agent can include, but is not limited to, 1) a biocompatible adhesive, 2) agents that promote endotheliazation of one or more portions of the medical device to thereby cause at least a portion of the medical device to be incorporated into the body passageway, and/or 3) abluminal coatings that promote adhesion of one or more portions of the medical device to the body passageway. In one non-limiting embodiment of the invention, one or more adhesion agents are located on the protective and/or supportive skin of the medical device so that the protective and/or supportive skin at least partially adheres to the diseased area on the body passageway when the medical device is at least partially expanded. This adhesion between the protective and/or supportive skin and the diseased area of the body passageway can be useful when the protective and/or supportive skin includes one or more chemical agents for healing and/or repairing the diseased area of the body passageway, since such adhesion will facilitate in directing the one or more chemical agent on the protective and/or supportive skin to the diseased area. In another and/or alternative non-limiting embodiment of the invention, one or more adhesion agents are located on one or more anchoring members and/or cross members of the medical device to promote the anchoring of the anchoring members and/or cross members to the body passageway when the medical device is at least partially expanded.

In still another and/or alternative non-limiting aspect of the invention, an inflation device (e.g., balloon, inflatable portion of a catheter, etc.) is used to at least partially expand one or more portions of the medical device when the medical device is position in the treatment area of the body passageway. The inflation device can be designed to apply an outwardly extending force from one or more portions of the interior region of the medical device when the inflation device is inflated so as to cause one or more portions of the medical device to expand. In one non-limiting embodiment of the invention, the medical device is at least partially crimped on an inflation device so as to reduce the size of the medical device to enable the medical device to be positioned at or near a diseased area of the body passageway. In another and/or alternative non-limiting embodiment of the invention, the inflation device is designed to expand one or more anchoring members of the medical device to cause the medical device to be at least partially anchored in the body passageway. In one non-limiting aspect of this embodiment, the inflation device is designed to cause a majority of the medical device to expand upon at least partial inflation of the inflation device. In another and/or alterative non-limiting aspect of this embodiment, the inflation device is designed to expand a different portion of the medical device in a different way. In one non-limiting design, the inflation device has a configuration that causes one or more anchoring members to expand at a greater rate and/or applies more force to the anchoring members than to the region between the anchoring members. This inflation device design facilitates in anchoring the medical device to the body passageway without causing the region between the anchoring members to injure or damage the diseased area on the body passageway. For instance, one or more inflation devices can be used to cause one or more anchoring members to be first expanded so as to at least partially anchor the medical device in a body passageway before partially or fully expanding the region between one or more anchoring members. As can be appreciated, many other inflation device configurations can be used to customize the expansion rates and/or force of expansion of one or more portions of the medical device when the inflation device is at least partially inflated. As also can be appreciated, more than one inflation device can be used to expand the medical device (i.e., a separate balloon for each anchoring member, a separate balloon for the region on the medical device spaced from one or more anchoring members, etc.).

In yet another and/or alternative non-limiting aspect of the invention, a sheath and/or an adhesive material can be used to control the expansion of one or more portions of the medical device when the medical device is a self expanding device. The sheath can be designed to partially or fully encircle the medical device. The sheath can be designed to be physically removed from the medical device after the medical device is deployed to a treatment area; however, this is not required. The sheath can be use in conjunction with one or more inflation devices to expand the medical device.

In one non-liming configuration of the medical device of the present invention, the medical device is designed to place a protective and/or supportive skin (e.g., a thin film or sheet, etc.) at least partially over a diseased area in a body passageway so as to prevent susceptible areas from rupturing, which can lead to blockage of the body passageway. Since the diseased area in the body passageway may be weakened, little or no direct force should occur directly at the sight of the diseased area at least until at least a portion of the diseased area is isolated from other portions of the body passageway; therefore, the medical device is designed to apply little or no stress on the diseased area at least until the medical device is at least partially anchored in the body passageway. This object is accomplished by the use of anchoring members on the medical device to be positioned at proximal and distal areas relative to the diseased area. Positioned between the anchoring members is a protective and/or supportive skin such as a thin sheet. The protective and/or supportive skin is designed to a) at least partially protect the diseased area, b) designed to deliver one or more chemical agents to the diseased area, which one or more chemical agents can be used to prevent susceptibility to rupture while resolving the cellular and molecular mechanisms which lead to the weakened/diseased status of the body passageway, and/or c) at least partially isolate the diseased area from other regions of the body passageway so that if one or more portions of the diseased area ruptures and/or fractures, the ruptures and/or fractures portions will be inhibited or prevent from traveling down the body passageway that is beyond the location of the medical device. The protective and/or supportive skin may also or alternatively contain surface molecules on the luminal side which promote endothelialization of the protective and/or supportive skin thereby resulting in the protective and/or supportive skin to be at least partially incorporated into the wall of the body passageway; however, this is not required. The protective and/or supportive skin may also or alternatively contain abluminal coatings that promote adhesion to the protective and/or supportive skin to the diseased area; however, this is not required. The protective and/or supportive skin can include one or more coatings, which one or more coatings can include one or more chemical agents, one or polymer layer and/or one or more metal layers; however, this is not required. The protective and/or supportive skin can be formed of a biostable or biodegradable polymer; however, this is not required. The protective and/or supportive skin can contain macro-, micro-, or nano-pores; however, this is not required. The protective and/or supportive skin can be 1 mm or less in thickness; however, other thicknesses can be used. The anchoring members on the medical can be or include circular members that expand to at least partially conform to the wall of the body passageway at points proximal and distal to the diseased area of the body passageway; however, other shapes can be used. The anchoring members can include micro or nano structures to aid in the anchoring the medical device to the non-diseased area of the body passageway; however, this is not required. The anchoring members can be connected by two or more cross members that are of sufficient length to transverse the diseased area of the body passageway; however, this is not required. When one or more cross members are used, the one or more cross members can be provided in varying lengths depending on the size of the diseased area of the body passageway; however, this is not required. The one or more cross members may include one or more expansion members; however, this is not required. The one or more cross members can be at least partially substituted by spiral members or a stent-type mesh, or the one or more cross members can be included with spiral members or a stent-type mesh; however, this is not required. The one or more cross members, spiral members and/or stent-type mesh at least partially form the skeleton of the medical device between the two anchoring members. The skeleton of the medical device and/or the anchoring members of the medical device can include metal alloy, and/or biostable or bioabsorbable polymer; however, this is not required. The protective and/or supportive skin can be at least partially adhered to the skeleton and/or anchoring members of the medical device by a medical adhesive, melting, etc., and/or the protective and/or supportive skin can be at least partially form as part of the skeleton; however, this is not required. The protective and/or supportive skin can exist on a fraction of the outer skeletal surface of the medical device (i.e., extend only about 20-60% about the circumference of the skeleton, extend only about 10-90% of the longitudinal length of the skeleton, etc.), or can be present about the entire skeletal outer surface and/or entire longitudinal length of the skeletal outer surface on the medical device. The medical device can be designed to be delivered on and be at least partially expanded by an inflation device (e.g., balloon member, inflatable catheter, etc.); however, this is not required. The inflation device can include one or more inflation portions over which the anchoring members of the medical device are at least partially mounted; however, this is not required. The anchoring members can be mechanically tightened (crimped) over the inflation device; however, this is not required. The inflation device can be used to expand the anchoring members at the proximal and/or distal areas outside of the diseased area of the body passageway and thereby cause the to be positioned and/or stretched over the diseased. During the positioning of the protective and/or supportive skin at least partially over a diseased area, the protective and/or supportive skin can be at least partially positioned over the diseased area in a manner so as to apply little, if any, pressure or any undue pressure to the diseased area which could result in damage, injury and/or rupture of the diseased area by the protective and/or supportive skin; however, this is not required. The inflation device material can include a biostable polymer currently used for angioplasty balloons; however, this is not required. The medical device can alternatively be self expanding for which a delivery sheath can be used for delivery and deployment of the medical device at a diseased area of the body passageway; however, this is not required. The delivery system for the medical device can includes radiopaque markers to identify the location of the anchoring members of the medical device as well as the skeleton (i.e. cross members, etc.) and/or protective and/or supportive skin of the medical device to aid in deployment of the medical device at a diseased area; however, this is not required. The radiopaque markers can include an alloy material as solid separate members or as painted members on the catheter assembly; however, this is not required. Radiopaque markers can also or alternatively exist on the skeleton member and/or anchoring members (e.g., applied to the surface and/or incorporated therein, etc.); however, this is not required. The radiopaque marker when in the form of bands can be applied over the delivery inflation device or sheath and/or applied directly to the medical device; however, this is not required. The configuration and/or positioning of the radiopaque markers with respect to the medical device and/or on the medical device can be used to assist in accurate placement of the medical device in the body passageway.

In another and/or alternative one non-liming configuration of the medical device of the present invention includes 1) a protective and/or supportive skin, 2) two circular anchoring members, 3) two or more cross members that support the protective and/or supportive skin and which the cross members are connected to the anchoring members, 4) an adhesive and/or one or more micro-structures on the anchoring members, the protective and/or supportive skin, and/or one or more cross members, 5) chemical agent on one or more anchoring members, the protective and/or supportive skin, and/or one or more cross members, and 6) a delivery system for the medical device that includes either an inflation device (e.g., balloon, inflatable catheter, etc.) or a sheath. In one non-limiting form of the medical device, the medical device includes two circular expandable anchoring members, a skeleton unit containing at least two cross members that are connected to the two anchoring members, and a protective and/or supportive skin is connected to the anchoring members and the cross members and which protective and/or supportive skin covers a portion or the entire outer circumference of the skeleton unit when the medical device is expanded. The medical device is designed to be mounted onto an inflation device in which at least a portion of the expandable portion of the inflation device is positioned under and/or is mounted to the anchoring members. The inflation device can be designed to exist as part of a catheter on which radiopaque markers exist at areas identifying the anchoring members and/or the cross members; however, this is not required. The protective and/or supportive skin is designed to be the portion of the medical device that is placed at least partially over the diseased area of the body passageway so as to a) inhibit or prevent damage to the diseased area (i.e., rupture of a vulnerable plaque, etc.), b) at least partially isolate the diseased portion from other portions of the body passageway, and/or c) deliver one or more chemical agents to the diseased area, when chemical agent is used. The adhesive and/or the micro-structures on the medical device, when used, are used to at least partially anchoring the medical device on the wall of the body passageway and/or at least partially secure the protective and/or supportive skin to the diseased area. The one or more chemical agents, when used, can include a substance that stimulates rather suppresses proliferation/activation. The anchoring members and cross members are designed to deliver the protective and/or supportive skin to the diseased area of the body passageway by acting as a scaffolding type arrangement that is anchored in areas proximal and distal to the diseased area on the body passageway. The inflation device is used to expand the anchoring members to ensure anchoring of the anchoring members to the wall of the body passageway without exerting direct pressure on the diseased area of the body passageway. When a delivery sheath is used to deliver the medical device in the body passageway, the sheath provides an enclosure for the collapsed state of the medical device while it is being delivered to the diseased area of the body passageway. The catheter is used to serve as a platform for the inflation device, when used, and radiopaque markers, when used, which markers allow for the proper placement of the protective and/or supportive skin over the diseased area without disrupting the diseased area. The radiopaque markers can be placed directly on or over the medical device, in which case the radiopaque marker serves the purpose of facilitating the accurate placement of the medical device and continues to function as locating point for any subsequent interventions in the body passageway; however, this is not required. The concept of placing a very protective and/or supportive skin with skeletal support at least partially over a diseased area and/or incorporating at least a portion of the protective and/or supportive skin into a diseased area of a body passageway (i.e., vulnerable plaque, etc.) is novel. The circular anchoring members, when used, represent configurations currently used for vascular grafts or stents. The inflation device, when used, can be similar to previously marketed balloons; however, a double inflation pattern (e.g., double balloon, etc.), when used, is a novel design. The use of a biological adhesive and/or the micro-structure, when used, to anchor the medical device in the wall of a body passageway is also novel. The use of radiopaque markers, when used, on one side of the medical device and/or along one or more cross members is also novel. The anchoring members and cross members allow delivery of the protective and/or supportive skin to a diseased area and enables the protective and/or supportive skin to be positioned over at least a portion of the diseased area on a body passageway without applying any or very little pressure to the diseased area, thereby inhibiting or prevent damage, injury and/or rupture of the diseased area when the medical device is expanded and anchored in the body passageway. When spiral cross members and/or expandable cross members are use in the skeleton of the medical device, these cross members can be used as supporting members to prevent the protective and/or supportive skin from collapsing. The inflation device, when used, and catheter enables the delivery and correct placement of the anchoring members in the body passageway. The inflation device can be eliminated when the anchoring members and/or skeleton are composed of a self-expanding material (e.g., metal alloy [e.g., Nitinol, etc.]), temperature sensitive polymers, etc.); however, this is not required. The anchor members can be absent micro-structures and/or adhesive when the anchoring members are self expanding; however, this is not required. When the skeleton includes a spiral or expanding support structures that are self expanding, an adhesive material and/or micro-structures on the protective and/or supportive skin surface can be eliminated; however, this is not required. When radiopaque markers are incorporated within the skeletal structure and/or anchoring members, the inflation device and/or catheter need not include such markers and/or additional markers need not be applied to the medical device; however, this is not required. The anchoring members, the skeleton and/or the protective and/or supportive skin can be composed of metal alloys, or biostable or biodegradable polymer; however, this is not required. One type of biodegradable polymer that can be used includes collagen which can promote healing of the ruptured fibrous cap of vulnerable plaque. The protective and/or supportive skin can be partially or completely replaced by a thin walled stent-like mesh which acts as a safety net to the diseased area on the body passageway and which will inhibit or prevent injury, damage and/or ruptured of the diseased area thereby inhibiting or preventing an undesired medical event (e.g., rupture of vulnerable plaque which thereby causes an embolic event, etc.); however, this is not required. The protective and/or supportive skin can include an adhesive material, in which case additional adhesives and/or micro-structure need not be used on the medical device; however, this is not required. The protective and/or supportive skin can have sufficient strength to tack up against the wall of the body passageway in which case the supporting skeletal structure can be eliminated; however, this is not required. A thin metallic film and/or polymer film can be deposited over the protective and/or supportive skin; however, this is not required. Layers of alternating films can be formed on the protective and/or supportive skin so as to render one or more regions of the sheet surface more inert or to promote a different type of cell activity on different regions of the protective and/or supportive film; however, this is not required. The protective and/or supportive film can have a thickness of less than or equal to about 1.0 mm; however, this is not required. As can be appreciated, more that one layer of protective and/or supportive film can be used on the medical device. The anchoring members are typically spaced from one another about 1-35 mm; however, this is not required. The anchoring members can be expanded to a diameter or maximum cross-section length of 1-13 mm; however, this is not required. The micro-structures, when used, have an average diameter or maximum cross-section length of about 0.1-1 mm and an average height of about 0.01-1 mm; however, this is not required. A wide variety of chemical agents, when used, can be used on and/or with the medical device. The dosage of chemical agent on the medical device is typically about 1 mg to 100 mg; however, this is not required. When the medical device is used in humans, the anchoring members, the skeleton and the protective and/or supportive film typically are made of FDA approved materials for human safety/use.

One non-limiting object of the present invention is the provision of a medical device that can at least partially isolate a diseased area in a body passageway from other regions of the body passageway.

Another and/or alternative non-limiting object of the present invention is the provision of a medical device that can be used to inhibit or prevent damage or rupturing of diseased areas of a body passageway.

Yet another and/or alternative non-limiting object of the present invention is the provision of a medical device that can be used to at least partially repair vulnerable plaque in a blood vessel.

Still another and/or alternative non-limiting object of the present invention is the provision of a medical device that includes one or more anchoring members used to at least partially secure the medical device at a location that is distal and/or proximal to a diseased area in a body passageway so as to limit or prevent damage or injury to the diseased area by the one or more anchoring members.

Yet another and/or alternative non-limiting object of the present invention is the provision of a medical device that can apply one or more thin films or sheets over at least a portion of a diseased area of a body passageway.

Still yet another and/or alternative non-limiting object of the present invention is the provision of a medical device that can apply one or more thin films or sheets over at least a portion of a diseased area of a body passageway with reduced force as compared to the anchor location of the medical device that are located proximal and/or distal to the ends of the diseased area of a body passageway.

A further and/or alternative non-limiting object of the present invention is the provision of a medical device that can apply one or more chemical agents to a diseased area of a body passageway to facilitate in the repair and/or healing of the diseased area of a body passageway.

Still a further and/or alternative non-limiting object of the present invention is the provision of a medical device that includes one or more micro-structures that are used to facilitate in the anchoring of the medical device to the body passageway.

Yet a further and/or alternative non-limiting object of the present invention is the provision of a medical device that includes one or more micro-structures that are used to facilitate in the insertion of one or more chemical agents in the diseased area of a body passageway and/or a region about the diseased area of a body passageway.

Still yet a further and/or alternative non-limiting object of the present invention is the provision of a medical device that includes an adhesive and/or other type of bonding material use to at least partially secure one or more portions of the medical device to the diseased area of a body passageway and/or a region about the diseased area of a body passageway.

These and other advantages will become apparent to those skilled in the art upon the reading and following of this description taken together with the accompanying drawings.

### BRIEF DESCRIPTION OF THE DRAWINGS

Reference may now be made to the drawings, which illustrate various embodiments that the invention may take in physical form and in certain parts and arrangements of parts wherein:

FIG. 1 is an elevation view of one non-limiting embodiment of the medical device in accordance with the present invention;

FIG. 2 is a sectional view of a blood vessel that includes a diseased area such as vulnerable plaque;

FIG. 3 is a sectional view of a blood vessel having the medical device of FIG. 1 positioned in the blood vessel and positioned over the diseased section of the blood vessel;

FIG. 3A is a sectional view of the blood vessel having the medical device of FIG. 1 wherein the protective and/or supportive film is positioned against the wall of the blood vessel;

FIG. 4 is a cross-sectional view along line 4-4 of FIG. 3;

FIG. 5A is a sectional view of a blood vessel and a crimped version of the medical device of FIG. 1 being delivered in the blood vessel by a catheter;

FIG. 5B is a sectional view of a blood vessel and the medical device of FIG. 1 being expanded in the blood vessel by a catheter;

FIG. 5C is a sectional view of a blood vessel and the medical device of FIG. 1 in an expanded state and the catheter being withdrawn from the medical device;

FIG. 6 is an elevation view of another non-limiting embodiment of the medical device in accordance with the present invention;

FIG. 7 is across-sectional view along line 7-7 of FIG. 6;

FIG. 8A-C are cross-sectional views of the medical device similar to FIG. 1 wherein the protective and/or supportive film is positioned in the inside surface of the medical device, between the cross members of the medical device, and about the cross members of the medical device;

FIG. 9 is an elevation view of another non-limiting embodiment of the medical device in accordance with the present invention; and,

FIGS. 10-10C is a cross-sectional view along line 10-10 of FIG. 9 which illustrates various positioning of the protective and/or supportive film on the medical device.

### DETAILED DESCRIPTION OF THE INVENTION

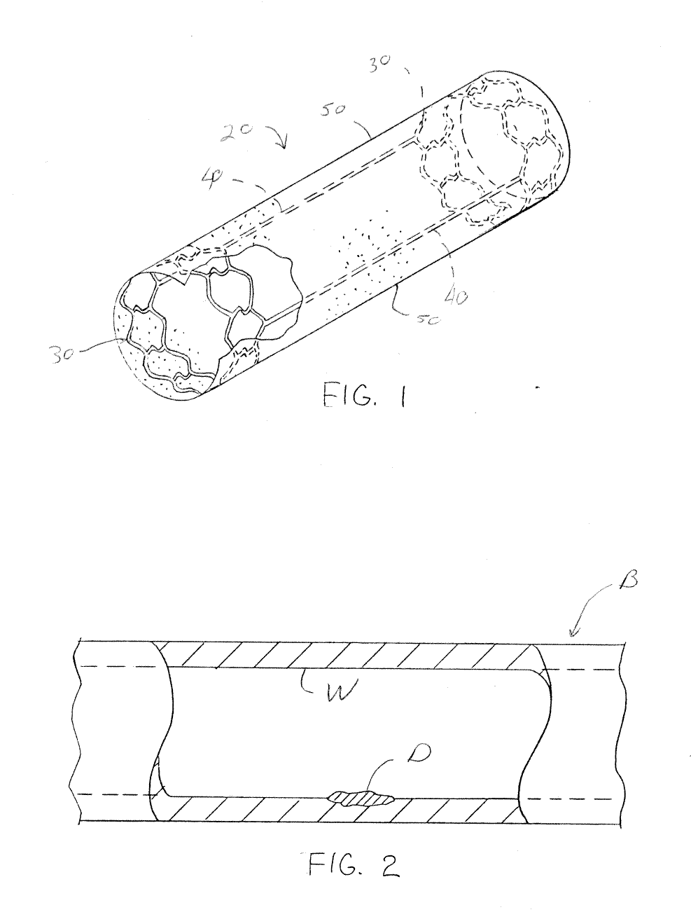

Referring now to the drawings wherein the showings are for the purpose of illustrating embodiments of the invention only and not for the purpose of limiting the same, FIG. 2 illustrates a blood vessel B that includes a diseased segment or area D such as vulnerable plaque on the interior wall W of the blood vessel; however, it will be appreciated that the diseased area can be a disease other than or in addition to vulnerable plaque. Although the invention will be described with particular reference to blood vessels, it will be appreciated that the medical device of the present invention can be used in other types of body passageways.

Referring again to FIG. 2, the vulnerable plaque D in the blood vessel B has a propensity to rupture when pressure is applied to the vulnerable plaque. Prior methods for treating blood vessels that were obstructed by plaque included the use of a stent or angioplasty balloon which were expanded in the location of the plaque to cause the plaque to be compressed to the wall of the blood vessel when the stent or angioplasty balloon was expanded. The force directed on the plaque by the stent or an angioplasty balloon risked rupture of the plaque, which rupture could result in the formation of a clot in the blood vessel and/or the dislodgement of some of the plaque which could then travel downstream of the diseased area in the blood vessel. The medical device of the present invention is designed to address the problems that can occur when a diseased area of a blood vessel or other type of body passageway is damaged or ruptured.

Referring now to FIG. 1, there is illustrated one non-limiting configuration of a medical device 20 of the present invention. The medical device 20 includes two anchoring members 30. These two anchoring members are illustrated as located at the two ends of the medical device. As can be appreciated, the medical device can include more than two anchoring members, and/or one or more of the anchoring members can be spaced from the ends of the medical device (e.g., positioned in the middle of the medical device, etc.). The medical device in FIG. 1 is illustrated to be in an expanded state. In the expanded state, the anchoring members are illustrated as generally circular in shape; however, it will be appreciated that one or more of the anchoring members can be expanded into other shapes (e.g., oval shaped, polygonal shaped, etc.). The configuration and dimensions of the anchoring members is non-limiting. Generally the two anchoring members have the same shape and size; however, this is not required. The two anchoring members are designed to facilitate in securing the medical device in a certain position in the blood vessel when one or more of the anchoring devices are expanded. This function of the anchoring members will be explained in more detail below. One or more of the anchoring members can include an adhesive and/or one or more micro-structures to facilitate in the connecting of one or more portions of the anchoring members to the inner wall of the blood vessel; however, this is not required. When an adhesive and/or one or more micro-structures are included on one or more anchoring members, the adhesive and/or one or more micro-structures can include one or more chemical agents; however, this is not required. One or more of the anchoring members can include a marker to facilitate in the positioning of the medical device in the blood vessel; however, this is not required.

Referring again to FIG. 1. the medical device 20 includes two cross members 40 that are connected to the two anchoring members 30. The cross members generally have a generally circular cross-sectional shape along the longitudinal length of the cross member; however, it will be appreciated that other cross-sectional shapes for the cross members can be used (e.g., oval, polygonal, etc.). Although only two cross members are illustrated, it will be appreciated that only one cross member can be used or more than two cross members can be used on the medical device (e.g., 3-10 cross members, etc.). The two cross members are illustrated as spaced at a generally equal distance from one another; however, this is not required. When three or more cross members are used, the cross members are generally equally spaced from one another and/or a plurality of sets of two cross members are spaced equal distances from one another; however, this is not required. One or more of the cross members can include an adhesive and/or one or more micro-structures to facilitate in the connecting of one or more portions of the cross members to the inner wall of the blood vessel; however, this is not required. When an adhesive and/or one or more micro-structures are included on one or more cross members, the adhesive and/or one or more micro-structures can include one or more chemical agents; however, this is not required. One or more of the cross members can include a marker to facilitate in the positioning of the medical device in the blood vessel; however, this is not required.

The cross members are designed to provide structural integrity and rigidity to the medical device. The cross members are also designed to provide support to the protective and/or supportive skin such as a thin film or sheet 50 on the medical device. When the medical device is expanded in a blood vessel, the one or more cross members on the medical device are used to at least partially move and maintain the protective and/or supportive film in contact with or in close proximity to the diseased portion of the body passageway. The protective and/or supportive film will be discussed in more detail below. The cross members can be made of the same or different material from the material used to form the anchoring members. The cross members can be designed to be more flexible and/or less rigid than the anchoring members; however, this is not required. As illustrated in the second figure of FIG. 1, the cross members are spaced apart a sufficient distance so that the cross members to not have to engage any portion or only a small portion of the diseased area on the body passageway when the anchoring members are expanded; however, this is not required. By minimizing or preventing contact of the cross members with the diseased area, the potential for injury, damage and/or rupture of the diseased area during the expansion of the medical device can be reduced, if so desired. The flexibility and rigidity of one or more of the cross members can also be selected to minimize or prevent injury, damage and/or rupture of the diseased area during the expansion of the medical device can be reduced, if so desired. The spacing of the cross members from one another can also be selected to minimize or prevent injury, damage and/or rupture of the diseased area during the expansion of the medical device can be reduced, if so desired.

Referring again to FIG. 1, a protective and/or supportive in the form of a thin film or thin sheet 50 is positioned about the outer surface of the anchoring members 30 and the cross members 40. As mentioned above, one or more portions of the thin sheet can be secured to one or more anchoring members and/or the cross members. In the non-limiting embodiment illustrated in FIG. 1, the thin sheet is only secured to the two anchoring members. As can be appreciated, the thin sheet can also or alternatively be secured to the cross members. The thin sheet can be secured to the anchoring members and/or cross members by a variety of mechanisms (e.g., adhesive, melt bond, mechanical connectors, etc.). As mentioned above, the anchoring members and/or cross members can be used to provide support to the thin sheet to facilitate in maintaining the thin sheet on or in close proximity to the diseased portion of the body passageway after the anchoring members have been expanded. The medical device can include additional structural supports, not shown, that can be used in conjunction with the thin sheet to facilitate in maintaining the thin sheet on or in close proximity to the diseased portion of the body passageway after the anchoring members have been expanded. The thin sheet is illustrated as extending beyond the ends of the two anchoring members; however, this is not required. As can be appreciated, the end of the thin sheet can be flush with the ends of one or more anchoring members or end short of the ends of one or more anchoring members. The thin sheet typically has a thickness that is no more than about 1 mm when the medical device is used to treat vulnerable plaque in a blood vessel; however, other thicknesses can be used. The thin sheet is also designed to be a more flexible and less rigid structure than the anchoring members no as to not apply undue force to the diseased area of the body passageway when the anchoring members are expanded, when such undue force is not desired. The thin sheet can further be designed to be a more flexible and less rigid structure than the cross members; however, this is not required. The thin sheet can be formed of a variety of materials such as, but not limited to, polymers. The physical properties of the thin sheet can be selected so as to avoid applying an undesired amount of force or pressure on the diseased area of the body passageway when the anchoring members are expanded, thereby inhibiting or preventing injury, damage and/or rupture of the diseased area. As can be appreciated, the thin sheet can be designed to be as strong, rigid, and/or flexible as the anchoring members and/or the cross members. The thin sheet can include one or more pores to allow for fluid flow through one or portions of the thin sheet; however, this is not required. The thin sheet can include an adhesive and/or one or more micro-structures to facilitate in the connecting of one or more portions of the thin sheet to the diseased area and/or inner wall of the blood vessel; however, this is not required. The thin sheet can include one or more micro-structures to facilitate in the connecting of one or more portions of the thin sheet to the inner wall of the blood vessel; however, this is not required. When an adhesive and/or one or more micro-structures are included on the thin sheet, the adhesive and/or one or more micro-structures can include one or more chemical agents; however, this is not required. The thin sheet can include one or more chemical agents on one or more portions of sides of the thin sheet. For example, the thin sheet can include one or more chemical agents used to promote endothelialization of the sheet so as to at least partially incorporate the thin sheet into the wall of the body passageway; however, this is not required. In another or additional example, the thin sheet can contain abluminal coatings that promote adhesion to the diseased area; however, this is not required. The thin sheet can include macro-, micro-, or nano-pores; however, this is not required. The thin sheet can include one or more layers of material so as to render one or more surfaces on the thin sheet more inert or to promote a different type of cell activity on one or more regions of the thin sheet; however, this is not required.

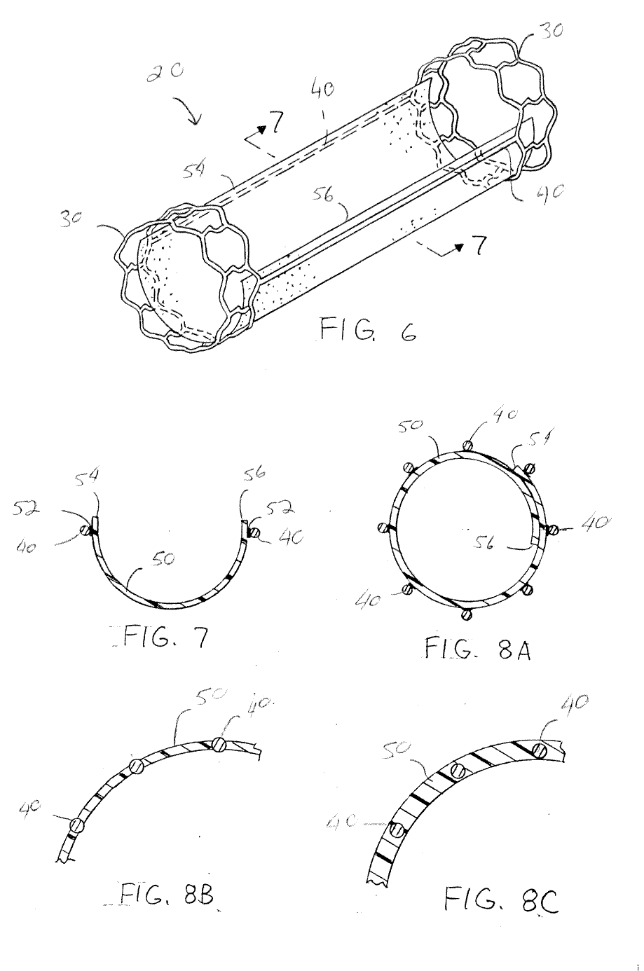

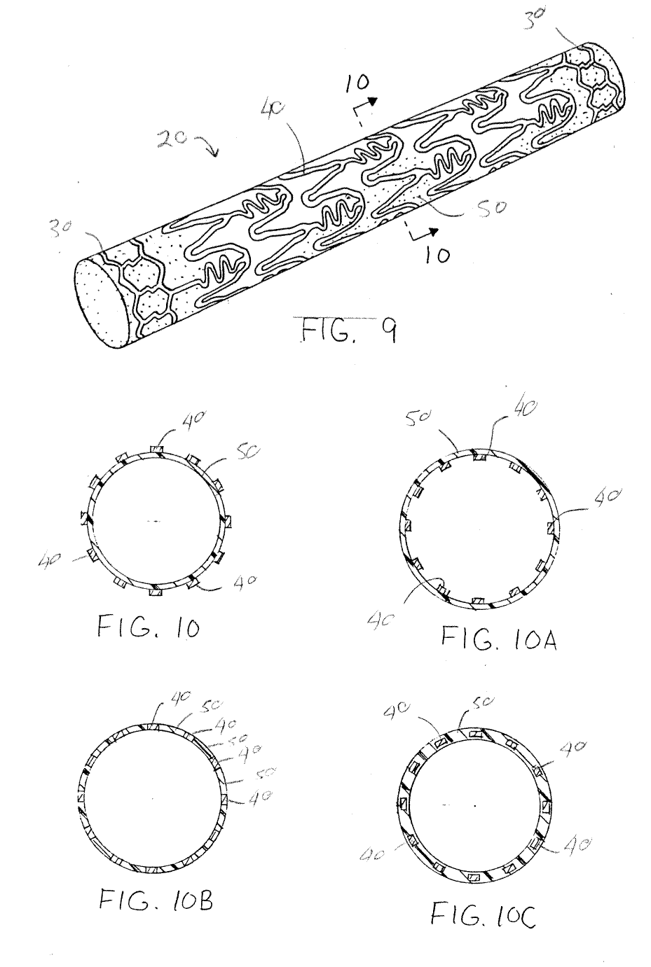

As mentioned above, the medical device illustrated in FIG. 1 represents just one of many embodiments of the medical device in accordance with the present invention. Several other non-limiting embodiments of the invention are illustrated in FIGS. 6-10C. The medical device 20 illustrated in FIG. 6 is similar in configuration as the medical device illustrated in FIG. 1 except for the configuration and attachment location of thin sheet 50. As illustrated in FIG. 6, the medical device includes two anchoring members 30 that are connected together by two cross members 40. As illustrated in FIGS. 6 and 7, the thin sheet is positioned on the inside surface of the cross members and the anchoring members. As best illustrated in FIG. 7, the thin sheet is illustrated as connected by an adhesive, weld or melted connection 52 to cross members 40; however, this is not required. The thin sheet is illustrated as extending short of ends of the two anchoring members; however, this is not required. As can be appreciated, the end of the thin sheet can be flush with the ends of one or more anchoring members or extend beyond the ends of one or more anchoring members. The thin sheet illustrated in FIGS. 6 and 7 is shown to only encircle a little over half of the interior surface of the cross members and the anchoring members. As illustrated in FIG. 1, the thin sheet fully encircles the outer surface of the cross members and the anchoring members. Although not illustrated, it will be appreciated that the medical device illustrated in FIG. 1 could be modified so that the thin sheet only encircles a portion of the outer surface of the cross members and the anchoring members. (e.g., encircles 20-99% of the outer surface of the cross members and the anchoring members, etc.). With reference again to FIGS. 6 and 7, it will be appreciated that the thin sheet can be designed to encircle more or less of the inner surface of the cross members and the anchoring members than illustrated in FIGS. 6 and 7. Indeed, FIG. 8A illustrates that the thin sheet fully encircles the inner surface of the cross members and the anchoring members.

Referring again to FIG. 8A, the medical device is illustrated as having more than two cross members 40. Eight cross members are illustrated in FIG. 8A; however, it can be appreciated that more or less cross members can be included on the medical device. FIG. 8A also illustrates the end edges 54, 56 of the thin sheet overlapping one another. It will be appreciated that the overlapping of the end edges of the thin sheet are not required. The overlapping portions can be secured together by an adhesive, melted connection, weld, etc.; however, this is not required.

Referring now to FIGS. 8B and 8C, there is illustrated two alternative embodiments to at least partially secure the thin sheet to the cross members and/or anchoring members. In FIGS. 8B, the thin sheet 50 is illustrated as connected to the sides of the cross members. The connection of the thin sheet to the cross members can be by an adhesive, melted connection, weld, etc.; however, other or additional types of connections can be used. As can be appreciated, the thin sheet can also be connected to the sides of the anchoring members; however, this is not required. In FIG. 8C, the thin sheet is shown to encapsulate the cross members. Such encapsulation can be accomplished by a dipping the cross members and/or anchoring members into a liquid material that ultimately dies to form the thin sheet, melting the thin sheet onto the cross members and/or anchoring members, etc. As can be appreciated, the thin sheet can be connected to one or more cross members and/or one or more anchoring members in different ways. For example, the thin sheet could be secured to the cross members as illustrated in FIG. 8C and be connected to the anchoring members as illustrated in FIG. 1. As can be appreciated, many other combinations for securing the thin sheet to the one or more cross members and/or one or more anchoring members can be used.

Referring now to FIGS. 9 and 10-10C, another non-limiting embodiment of the medical device is illustrated. As illustrated in FIG. 9, medical device 20 includes two anchoring members 30 and a single cross member 40 connected between the two anchoring members. A thin sheet 50 is positioned about the outer surface of the cross member 40 and two anchoring members 30. As mentioned above, the thin sheet can be positioned only about a portion of the cross member 40 and two anchoring members 30, and/or be positioned on various regions on the anchoring members. The single cross member is illustrated to be in the form of a mesh material. The configuration of the cross member is such that it encircles the perimeter of the medical device. As can be appreciated, the mesh design of the cross member can be designed to encircle only a portion of the perimeter of the medical device. As can also be appreciated, multiple types of cross members can be used on the medical device. For example, one or more cross members can have a mesh configuration and one or more cross member can have a rod shaped configuration. As illustrated in FIGS. 9 and 10A, the thin sheet 50 is positioned about the outer side surface of the cross member 40 and the anchoring members 30. As illustrated in FIGS. 10, 10B and 10C, the thin sheet can be oriented on the cross member and the anchoring members in other or additional ways. As illustrated in FIG. 10, the thin sheet 50 is connected to the inner surface of the cross member and the anchoring members. In FIG. 10B, the thin sheet is connected to the sides of the cross member and the anchoring members. In FIG. 10C, the thin sheet at least partially encapsulates the cross member and the anchoring members. As can be appreciated other or additional configurations for the connection of the thin sheet to the cross member and/or the anchoring members can be used.

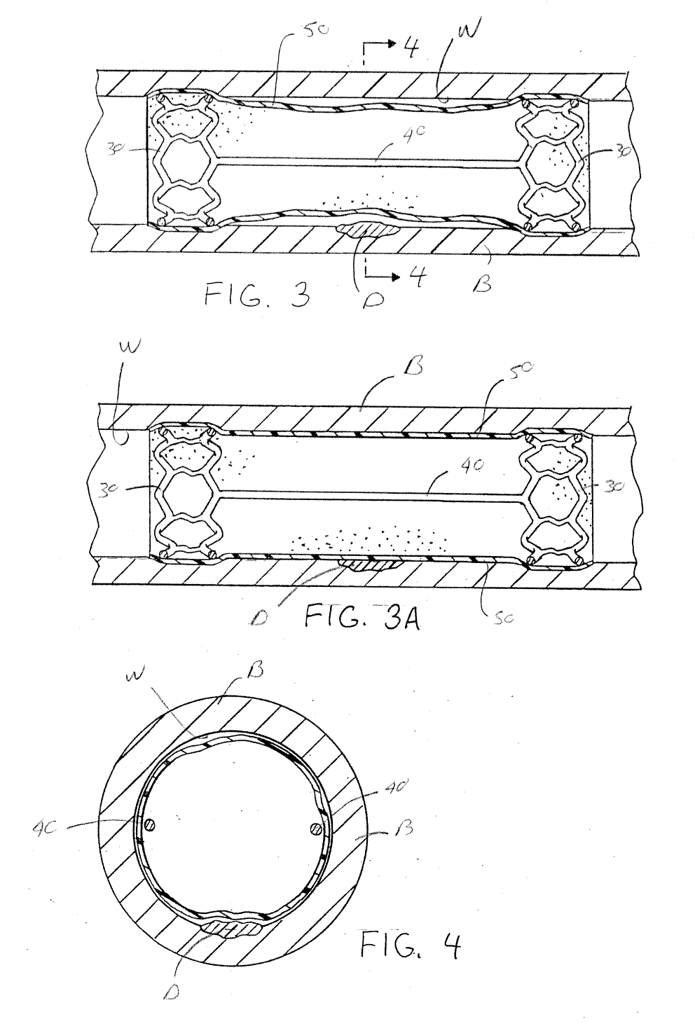

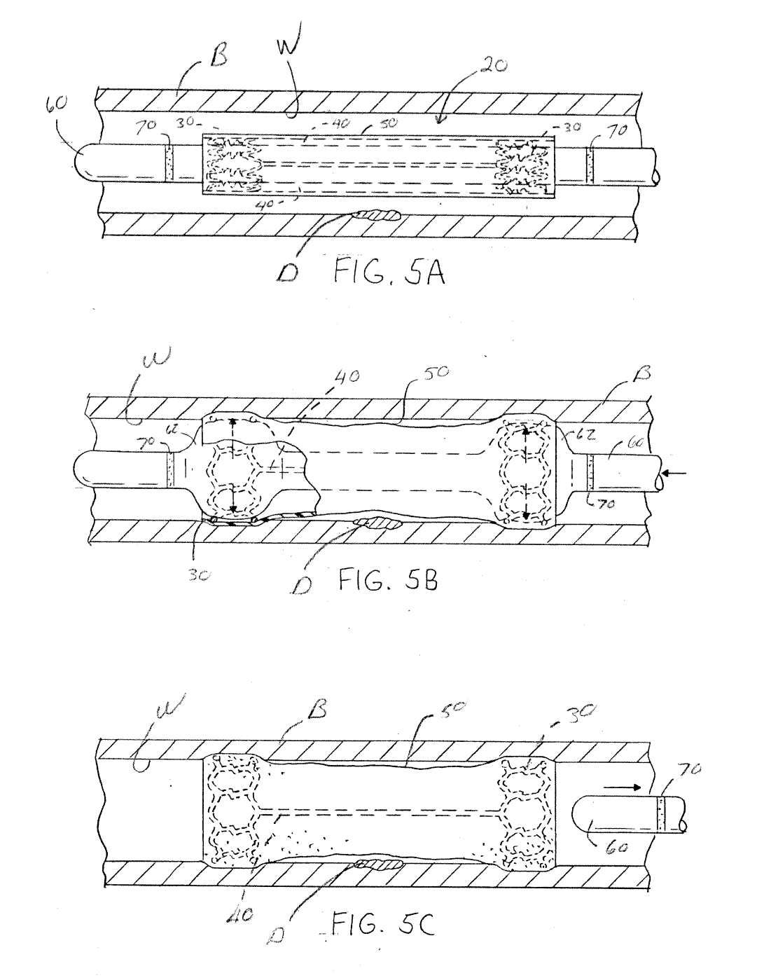

One non-limiting methodology for inserting the medical device 20 into a blood vessel is illustrated in FIG. 3-5C. As illustrated in FIG. 5A, a catheter 60 is used to position the medical device 20 in blood vessel B. The catheter can be standard type of catheter that is well known in the art. The catheter 60 can include one or more markers 70 that are used to facilitate in the proper positioning of the medical device in the blood vessel. The medical device is shown in a crimped or unexpanded orientation on the catheter so as to enable the medical device to be positioned in the blood vessel. As illustrated in FIG. 5A, the catheter positions the medical device so that the anchoring members 30 on the medical device are positioned on both sides of the diseased area D in the blood vessel.

Once the medical device is properly positioned in the blood vessel, the catheter is used to expand the medical device. As illustrated in FIG. 5B, the catheter is used to expand the anchoring members so as to secure the medical device in the blood vessel. As illustrated in FIG. 5B, the two anchoring members 30 are spaced a sufficient distance apart from each another so that when the anchoring members are expanded, the anchoring members are spaced from the distal and proximal ends of the diseased area D on the blood vessel. The spacing of the anchoring members from the diseased area in the blood vessel results in the anchoring members not applying a compressive force on or near the diseased area, which compressive force could result in injury, damage and/or rupture of the diseased area. For medical devices used in blood vessels, the distance between the two anchoring members is generally about 1-35 mm. The spacing of the anchoring members from each other will generally depend on the size of the diseased area and the location of the diseased area in the blood vessel and the type of blood vessel; however, other or additional factors may also be used to determining such spacing.

As mentioned above, the medical device in FIG. 5A is shown to be crimped on a catheter which is used to insert the medical device in the body passageway. The catheter is shown to include two inflatable sections 62 that are integrated in the catheter. It can be appreciated that one or more of the inflatable sections need not be integrated with the catheter. The inflatable sections 62 of catheter 60 are positioned underneath the two anchoring members 30. As illustrated in FIG. 5B, the medical device is shown to be crimped on a catheter which is used to insert the medical device in the body passageway. The inflatable sections of the catheter are positioned underneath the two anchoring members. This inflatable catheter configuration is designed to only apply an expansion force to the anchoring members when the medical device is expanded. This controlled application of expansion force on the medical device facilitates in ensuring that no undue force or pressure is applied by the medical device on the wall of the body passageway where the diseased area is located. As can be appreciated, the inflatable catheter can be designed so that some expansion force is applied to the medical device in a region between the anchoring members; however, this is not required. The controlled application of expansion force on the medical device facilitates in ensuring that no undue force or pressure is applied by the medical device on the wall of the body passageway where the diseased area is located. As can be appreciated, the inflatable catheter can be designed so that some expansion force is applied to the medical device in a region between the anchoring members; however, this is not required.

As illustrated in FIGS. 5B and 5C, the shape of the anchoring members can be similar to the end portions of commonly used stents or can be different. As can be appreciated, the structure and/or materials used to form the anchoring members can be the same or similar to the end sections of various types of stents such as, but not limited to, stents illustrated in U.S. Pat. No. 6,206,916; U.S. Pat No. 6,436,133; U.S. Pat. No. 6,974,475; US 2004/0093076; US 2004/0093077; and all the prior art cited in these patents and patent publications. The anchoring members are made of a material and have a structure that enables the outer surface of the anchoring members to engage the inner wall of the body passageway with sufficient force to retain the medical device in position after the anchoring members have been expanded. The materials used to form the anchoring members can be biostable or biodegradable. Non-limiting examples of materials that can be used to form the anchoring members includes metals or polymers. As the anchoring members are expanded by the two inflatable sections, the two cross members also begin moving toward the inner wall surface of the body passageway. The movement of the cross members and/or the expansion of the anchoring members causes the thin sheet to move toward the inner wall surface of the body passageway and to eventually cover all or a portion of the diseased area in the body passageway. The thin sheet can include one or more pores; however, this is not require. The pores in the thin sheet, when used, can be used to facilitate in the movement of the thin sheet toward the inner wall surface of the body passageway; however, the pores can have other or additional functions.

Once the medical device is expanded, the inflatable catheter deflated and withdrawn from the blood vessel. As illustrated in FIGS. 3, 4 and 5C, the thin sheet 50 is positioned on or in very close proximity to the diseased area after the anchoring members have been expanded. FIG. 3A illustrates the thin sheet 50 positioned against the wall of the blood vessel after the anchoring members have been expanded. In this non-limiting embodiment, the thin sheet is formed of a less flexible material, thus when the anchoring members are expanded, the thin sheet moves with the expanding anchoring members until the thin sheet engages the wall of the blood vessel.

As described in detail above, the medical device is used to place a thin film or sheet over at least a portion of a diseased area in a body passageway such as, but not limited to, blood vessels to inhibit or prevent susceptible or diseased areas on the body passageway from rupturing, which in turn could lead to blockage of the body passageway. Since the diseased area of the body passageway is typically weakened, the medical device of the present invention is designed to exert little or no direct force on the diseased area during the placement and expansion of the anchoring members of the medical device in the blood vessel. This is in part accomplished by designing a medical device that includes anchoring members which are designed to engage the wall of the body passageway at a location that is spaced from the proximal and distal areas of the diseased area of the body passageway. The medical device also includes a thin sheet positioned between the anchoring members that is designed to at least partially protect the diseased area. The thin sheet can include one or more chemical agents that are selected to at least partially heal and/or repair the diseased area of the body passageway. Once the anchoring members have been expanded, the middle portion of the medical device can be expanded; however, this is not required. In the embodiments of the medical device illustrated in FIGS. 1, 3 and 6, the expansion of the anchor members causes the cross members to move toward the inner wall of the blood vessel and cause the thin sheet to be positioned on or close to the diseased area in the blood vessel. The medical device illustrated in FIG. 9 is designed to generally require the middle portion of the medical device to be expanded one the anchoring members have been expanded so as to cause the thin sheet in the middle portion of the medical device to be moved on or in closed proximity to the inner wall of the blood vessel. A similar positioning of the thin film can also occur by use of the medical device illustrated in FIGS. 9, 10A-10C. When the cross member 40 is formed of a relatively rigid material, the cross member can be moved or forced into engagement with the wall of the blood vessel, thereby causing the thin sheet to engage the wall of the blood vessel, irrespective of whether the thin sheet is as rigid or less rigid than the anchoring members and/or the cross member.

In all of the embodiments of the medical device, the expansion of the anchoring members in combination with the movement of the cross member(s) and thin sheet on or close proximity to the inner wall of the blood vessel results in the partial or complete covering and isolation of the diseased portion from other regions of the blood vessel. As such, if one or more portions of the diseased ear becomes dislodged, the thin sheet inhibits or prevents the dislodge portion of the diseased area from escaping from the medical device.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

1-21. (canceled)

22. A method of treating a body passageway that includes a diseased area comprising the steps of:
a. providing a medical device that includes first and second expandable ends and a film connected to at least a portion of said expandable ends, said film being a protective film, a supportive film, or combinations thereof, said first and second expandable ends connected together by a plurality of cross members, at least one of said cross members connected to both said first and second expandable ends, said first and second expandable ends having a different structural configuration from said plurality of cross members, said first and second expandable ends spaced apart from one another by said plurality of cross members;
b. positioning said medical device in the body passageway such that said first expandable end is positioned on one side of the diseased area in the body passageway and said second expandable end is positioned on an opposite side of the diseased area in the body passageway; and,
c. applying a radial force to said first and second expandable ends to sufficiently expand said first and second expandable ends so that said first and second expandable ends engage an inner surface of said body passageway and maintain said medical device in position relative to said medical device, said expanded first expandable end being positioned at a location that is proximal to said diseased area and said expanded second expandable end being positioned at a location that is distal to said diseased area and said film at least partially overlying at least a portion of said diseased area to thereby encapsulate at least a portion of said diseased area between said inner surface of said body passageway so as to inhibit or prevent a portion of said diseased area form breaking off and flowing down through said body passageway, said first and second expandable ends, said plurality of cross members and said film, said first and second expandable ends expanded at a different rate, to a different degree, or combinations thereof from said cross members when said first and second expandable ends are expanded to anchor said medical device in a location in said body passageway.

23. The method as defined in claim 22, wherein at least two of said cross members are positioned along a longitudinal axis of said stent, at least one of said cross members is connected to both of said first and second expandable ends, at least two of said cross members are spaced from one another along the complete longitudinal length of said stent.

24. The method as defined in claim 22, wherein at least one of said cross members is a substantially straight member that connects to both of said first and second expandable ends, at least two of said cross members are spaced from one another along the complete longitudinal length of said stent, at least one of said first and second expandable ends encircling the inner surface OF a portion of the body passageway when at least one of said first and second expandable ends is expanded.

25. The method as defined in claim 23, wherein at least one of said cross members is a substantially straight member that connects to both of said first and second expandable ends, at least two of said cross members are spaced from one another along the complete longitudinal length of said stent, at least one of said first and second expandable ends encircling the inner surface of a portion of the body passageway when at least one of said first and second expandable ends is expanded.

26. The method as defined in claim 22, further including the step of first expanding at least a portion of said first expandable end, second expandable ends, or combinations thereof to at least partially anchor said stent in the body passageway and to inhibit or prevent said diseased area from breaking off and flowing down through said body passageway prior to said cross members being expanded, and then subsequently expanding other portions of said stent until said stent is fully expanded in the body passageway and a desired portion of the diseased area is at least partially encapsulated between the inner surface of the body passageway and said film.

27. The method as defined in claim 24, further including the step of first expanding at least a portion of said first expandable end, second expandable ends, or combinations thereof to at least partially anchor said stent in the body passageway, and then subsequently expanding other portions of said stent until said stent is fully expanded in the body passageway and a desired portion of the diseased area is at least partially encapsulated between the inner surface of the body passageway and said film.

28. The method as defined in claim 25, further including the step of first expanding at least a portion of said first expandable end, second expandable ends, or combinations thereof to at least partially anchor said stent in the body passageway, and then subsequently expanding other portions of said stent until said stent is fully expanded in the body passageway and a desired portion of the diseased area is at least partially encapsulated between the inner surface of the body passageway and said film.

29. The method as defined in claim 22, wherein at least one of said first and second expandable ends, at least one of said cross members, said film, or combinations thereof include a plurality of micro-structures, a plurality of said micro-structures including a chemical agent, and including the step of causing a plurality of said micro-structures to penetrate into the inner surface of said body passageway that includes the diseased region, does not include the diseased region, or combinations thereof when said stent is expanded in the body passageway, said chemical agent formulated to treat the diseased area in the body passageway.

30. The method as defined in claim 27, wherein at least one of said first and second expandable ends, at least one of said cross members, said film, or combinations thereof include a plurality of micro-structures, a plurality of said micro-structures including a chemical agent, and including the step of causing a plurality of said micro-structures to penetrate into the inner surface of said body passageway that includes the diseased region, does not include the diseased region, or combinations thereof when said stent is expanded in the body passageway, said chemical agent formulated to treat the diseased area in the body passageway.

31. The method as defined in claim 28, wherein at least one of said first and second expandable ends, at least one of said cross members, said film, or combinations thereof include a plurality of micro-structures, a plurality of said micro-structures including a chemical agent, and including the step of causing a plurality of said micro-structures to penetrate into the inner surface of said body passageway that includes the diseased region, does not include the diseased region, or combinations thereof when said stent is expanded in the body passageway, said chemical agent formulated to treat the diseased area in the body passageway.

32. The method as defined in claim 22, wherein said structural configuration of said first and second expandable ends is designed to contact a complete inner perimeter surface of the body passageway when said first and second expandable ends are expanded in the body passageway, said structural configuration of a plurality of said cross members designed to not contact a complete inner perimeter surface of the body passageway when said medical device is fully expanded in the body passageway.

33. The method as defined in claim 30, wherein said structural configuration of said first and second expandable ends is designed to contact a complete inner perimeter surface of the body passageway when said first and second expandable ends are expanded in the body passageway, said structural configuration of a plurality of said cross members designed to not contact a complete inner perimeter surface of the body passageway when said medical device is fully expanded in the body passageway.

34. The method as defined in claim 31, wherein said structural configuration of said first and second expandable ends is designed to contact a complete inner perimeter surface of the body passageway when said first and second expandable ends are expanded in the body passageway, said structural configuration of a plurality of said cross members designed to not contact a complete inner perimeter surface of the body passageway when said medical device is fully expanded in the body passageway.

35. The method as defined in claim 22, wherein said stent has an outer surface and an inner surface that defines an inner surface of a cavity of said stent, said film positioned on said inner surface, said outer surface of said stent, or combinations thereof.

36. The method as defined in claim 35, wherein said film is positioned on said outer surface of said stent.

37. The method as defined in claim 33, wherein said stent has an outer surface and an inner surface that defines an inner surface of a cavity of said stent, said film positioned on said outer surface of said stent.

38. The method as defined in claim 34, wherein said stent has an outer surface and an inner surface that defines an inner surface of a cavity of said stent, said film positioned on said outer surface of said stent.

39. The method as defined in claim 22, wherein said film encircles a region of said stent that is positioned between said first and second expandable ends.

40. The method as defined in claim 37, wherein said film encircles a region of said stent that is positioned between said first and second expandable ends.

41. The method as defined in claim 38, wherein said film encircles a region of said stent that is positioned between said first and second expandable ends.

42. The method as defined in claim 22, including a chemical agent positioned on said first expandable end, said second expandable end, said cross members, said film, or combinations thereof.

43. The method as defined in claim 40, including a chemical agent positioned on said first expandable end, said second expandable end, said cross members, said film, or combinations thereof.

44. The method as defined in claim 41, including a chemical agent positioned on said first expandable end, said second expandable end, said cross members, said film, or combinations thereof.
